SEQUENCE LISTING (18 sequences)

SEQ ID NO: 1 - protein, 314 aa, Simonsiella muelleri
  source 1..314: note = ATCC 29453
MKNLFICLTPLQSLIAQSLIRKTAPTPADLLMICYAETDNAKFRHYYQETAQLCQYAQFIRIPQNTWLREITLPFITKNLSKQYDTIYAASIDNPNVQYPLSHLSFKKLETFDDGTANLYPDSILYQNQKYGKKSKIIRKLQGICYTTEDLRKLSSAHHTLYPTQKNIVSPTIALKLWQNSAFLSDSGSLKTQKILLGQPLFDETADNISLFQSLLQCTQADAYFPHPRESYRLLNANYIDTPLIFEDYLLKRIQQEPHTKFHIYHVASTAALNVVGFPHTQIHAIRPPHALFQRESFNYLYDLMAKMNIEIQT

SEQ ID NO: 2 - protein, 301 aa, Zymomonas mobilis
MKNDKKIYGCIEGGGTKFMLALIDSDRKMLAVERVPTTTPEETLGKSVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 3 - protein, 504 aa, Bifidobacterium adolescentis [sequence omitted]

SEQ ID NO: 4 - protein, 417 aa, Escherichia coli [sequence omitted]

SEQ ID NO: 5 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 6 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 7 - protein, 349 aa, Neisseria meningitidis
MQNNNEFKIGNRSVGYNHEPLIICEIGINHEGSLKTAFEMVDAAYNAGAEVVKHQTHIVEDEMSDEAKQVIPGNADVSIYEIMERCALNEEDEIKLKEYVESKGMIFISTPFSRAAALRLQRMDIPAYKIGSGECNNYPLIKLVASFGKPIILSTGMNSIESIKKSVEIIREAGVPYALLHCTNIYPTPYEDVRLGGMNDLSEAFPDAIIGLSDHTLDNYACLGAVALGGSILERHFTDRMDRPGPDIVCSMNPDTFKELKQGAHALKLARGGKKDTIIAGEKPTKDFAFASVVADKDIKKGELLSGDNLWVKRPGNGDFSVNEYETLFGKVAACNIRKGAQIKKTDIE

SEQ ID NO: 8 - protein, 609 aa, Escherichia coli
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 9 - protein, 188 aa, Escherichia coli
MYERYAGLIFDMDGTILDTEPTHRKAWREVLGHYGLQYDIQAMIALNGSPTWRIAQAIIELNQADLDPHALAREKTEAVRSMLLDSVEPLPLVDVVKSWHGRRPMAVGTGSESAIAEALLAHLGLRHYFDAVVAADHVKHHKPAPDTFLLCAQRMGVQPTQCVVFEDADFGIQAARAAGMDAVDVRLL

SEQ ID NO: 10 - protein, 373 aa, Campylobacter jejuni
MVKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 11 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

SEQ ID NO: 12 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 13 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 14 - protein, 445 aa, Escherichia coli
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 15 - protein, 456 aa, Escherichia coli
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 16 - protein, 346 aa, Campylobacter jejuni
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 17 - protein, 415 aa, Escherichia coli
  source 1..415: note = W
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 18 - protein, 498 aa, Photobacterium sp.
  source 1..498: note = JT-ISH-224
MSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

(19) United States
(12) Patent Application Publication
(10) Pub. No.: US 2025/0043253 A1
(43) Pub. Date: Feb. 6, 2025

(54) SIALYLTRANSFERASES FOR THE PRODUCTION OF SIALYLATED OLIGOSACCHARIDES

(71) Applicant: INBIOSE N.V., Zwijnaarde (BE)

(72) Inventors: Joeri Beauprez, Zwijnaarde (BE); Thomas Decoene, Zwijnaarde (BE); Annelies Vercauteren, Zwijnaarde (BE); Tom Verhaeghe, 9052 (BE)

(21) Appl. No.: 18/717,393

(22) PCT Filed: Dec. 15, 2022
(86) PCT No.: PCT/EP2022/086076

§ 371 Date: Jun. 6, 2024

(30) Foreign Application Priority Data
Dec. 15, 2021 (EP) 21214943.9
Dec. 16, 2021 (EP) 21215198.9

(51) Int. Cl. C12N 9/10; C12P 19/26

(52) U.S. Cl. CPC C12N 9/1081; C12P 19/26

(57) ABSTRACT

Newly identified alpha-2,6-sialyltransferases are disclosed herein. The disclosure also provides for production of sialylated di- and/or oligosaccharides and relates to the use of the sialyltransferases in such methods and cells. The disclosure is also in the technical field of synthetic biology and metabolic engineering. More particularly, the disclosure is in the technical field of metabolically engineered cells and use of the cells in a cultivation or incubation. The disclosure describes a metabolically engineered cell and a method by cultivation or incubation with the cell for production of 6′sialylated disaccharide and/or 6′sialylated oligosaccharide. Furthermore, the disclosure provides for purification of the 6′sialylated disaccharide and/or 6′sialylated oligosaccharide from the cultivation or incubation.

### CROSS-REFERENCE TO RELATED APPLICATIONS

This application is a national phase entry under 35 U.S.C. § 371 of International Patent Application PCT/EP2022/086076, filed Dec. 15, 2022, designating the United States of America and published as International Patent Publication WO 2023/111141 A1 on Jun. 22, 2023, which claims the benefit under Article 8 of the Patent Cooperation Treaty of European Patent Application Serial No. 21214943.9, filed Dec. 15, 2021, and European Patent Application Serial No. 21215198.9, filed Dec. 16, 2021.

### STATEMENT REGARDING ELECTRONIC FILING OF A SEQUENCE LISTING

Pursuant to 37 C.F.R. § 1.834, a Sequence Listing XML file entitled “6W55877-P18056US-047-PCT-US sequence lis.xml,” 23.3 kilobytes in size, generated Jun. 6, 2024, has been submitted via EFS-Web and is provided in lieu of a paper copy. This Sequence Listing is hereby incorporated by reference into the specification for its disclosures.

### TECHNICAL FIELD

The disclosure relates to newly identified alpha-2,6-sialyltransferases. The disclosure also provides for production of sialylated di- and/or oligosaccharides and relates to the use of the sialyltransferases in such methods and cells. The disclosure is also in the technical field of synthetic biology and metabolic engineering. More particularly, the disclosure is in the technical field of metabolically engineered cells and use of the cells in a cultivation or incubation. The disclosure describes a metabolically engineered cell and a method by cultivation or incubation with the cell for production of 6′sialylated disaccharide and/or 6′sialylated oligosaccharide. Furthermore, the disclosure provides for purification of the 6′sialylated disaccharide and/or 6′sialylated oligosaccharide from the cultivation or incubation.

### BACKGROUND

More than 150 structurally distinct human milk oligosaccharides (HMOs) have been identified to date. Although HMOs represent only a minor amount of total human milk nutrients, their beneficial effects on the development of breast-fed infants became evident over the past decades.

Among the HMOs, sialylated HMOs (SHMOs) were observed to support several beneficial effects as described in the art. Among the sialylated di- and/or oligosaccharides in human milk, 3′sialyllactose, 6′sialyllactose, sialyllacto-N-tetraose a, sialyl lacto-N-tetraose b, sialyllacto-N-tetraose c and disialyllacto-N-tetraose are the most prevalent members.

Sialylated di- and/or oligosaccharides are found to be a complex structure and their chemical or (chemo-) enzymatic syntheses has been proven challenging: there are extensive difficulties, e.g., control of stereochemistry, formation of specific linkages, availability of feedstocks, etc. As a consequence, alternative production methods have been developed, amongst which efforts in metabolic engineering of microorganisms to produce sialylated di- and/or oligosaccharides have been made.

Several sialyltransferases have been identified and characterized to date, from bacterial species, e.g., from Neisseria, Campylobacter, Pasteurella, Helicobacter and Photobacterium, as well as from mammals and viruses. Sialyltransferases have been generally classified into six glycosyltransferase (GT) families, based on protein sequence similarities. Sialyltransferases are distinguished due to the glycosidic linkages that they form, e.g., into a-2,3-, a-2,6- and a-2,8-sialyltransferases. All of these sialyltransferases transfer the sialic acid residue from cytidine 5′-monophosphate sialic acid (e.g., CMP-NeuNAc) to a variety of acceptor molecules, usually a galactose (Gal) moiety, an N-acetylgalactosamine (GalNAc) moiety or an N-acetylglucosamine (GlcNAc) moiety or another sialic acid (Sia) moiety. Several bacterial a-2,6-sialyltransferases were well characterized in the past and are already proven to be suitable for the production of 6′sialyllactose (6′SL). The most commonly used enzymes for microbial 6′SL production originate from marine bacteria: Pst-6 of Photo bacterium sp. JT-ISH-224, St0160 of Photobacterium damselae JT0160, PlsT6 of Photo bacterium leiognathi JT-SHIZ-119 and PlsT6 of Photobacterium leiognathi JT-SHIZ-145.

### BRIEF SUMMARY

Provided herein are tools and methods by means of which a sialylated di- and/or oligosaccharide, like, e.g., 6′sialylated disaccharide and/or 6′sialylated oligosaccharide and 6′sialyllactose, can be produced, preferably in an efficient, time- and cost-effective way that yields high amounts of the desired oligosaccharide.

Provided is a newly identified alpha-2,6-sialyltransferase as described herein, which can be used in a method for the production of a 6′sialylated disaccharide and/or 6′sialylated oligosaccharide, preferably 6′sialyllactose. Furthermore, this newly identified alpha-2,6-sialyltransferase can be used in a cell for production of a 6′sialylated disaccharide and/or 6′sialylated oligosaccharide, preferably 6′sialyllactose.

More specifically, the disclosure provides a cell and a method for the production of a 6′sialylated disaccharide and/or 6′sialylated oligosaccharide, preferably 6′sialyllactose, wherein the cell is metabolically engineered with the alpha-2,6-sialyltransferase as described herein and comprises a pathway for the production of the 6′sialylated disaccharide and/or 6′sialylated oligosaccharide.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the disclosure described herein are to be understood not only in the order and context specifically described in this specification, but to include any order and any combination thereof. Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference in its entirety.

Whenever the context requires, unless specifically stated otherwise, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps, unless specifically stated otherwise.

Throughout the application, unless explicitly stated otherwise, the features “synthesize,” “synthesized” and “synthesis” are interchangeably used with the features “produce,” “produced” and “production,” respectively. Throughout the application, unless explicitly stated otherwise, the expressions “capable of . . . <verb>” and “capable to . . . <verb>” are preferably replaced with the active voice of the verb and vice versa. For example, the expression “capable of expressing” is preferably replaced with “expresses” and vice versa, i.e., “expresses” is preferably replaced with “capable of expressing.” In this document and in its claims, the verb “to comprise,” “to have” and “to contain” and their conjugations are used in their non-limiting sense to mean that items following the word are included, but items not specifically mentioned are not excluded. Throughout the application, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, the additional component(s) not altering the unique characteristic of the disclosure. In this document and in its claims, unless specifically stated otherwise, the verbs “to comprise,” “to have” and “to contain,” and their conjugations, may be replaced by “to consist of” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one.”

Throughout the application, unless explicitly stated otherwise, the articles “a” and “an” are preferably replaced by “at least two,” more preferably by “at least three,” even more preferably by “at least four,” even more preferably by “at least five,” even more preferably by “at least six,” and most preferably by “at least two.” The word “about” or “approximately” when used in association with a numerical value (e.g., “about 10”) or with a range (e.g., “about x to approximately y”) preferably means that the value or range is interpreted as being as accurate as the method used to measure it. If no error margins are specified, the expression “about” or “approximately” when used in association with a numerical value is interpreted as having the same round-off as the given value. Throughout this document and its claims, unless otherwise stated, the expression “from x to y,” wherein x and y represent numerical values, refers to a range of numerical values wherein x is the lower value of the range and y is the upper value of the range. Herein, x and y are also included in the range.

According to the disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The terms “recombinant” or “transgenic” or “metabolically engineered” or “genetically engineered” as used herein with reference to a cell or host cell are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene or are described to be transformed by the introduction of at least one heterologous or exogenous gene. Metabolically engineered or recombinant or transgenic or genetically engineered cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; CrispR; riboswitch; recombineering; ssDNA mutagenesis; transposon mutagenesis and related techniques as known to a person skilled in the art. Accordingly, a “recombinant polypeptide” is one that has been produced by a recombinant cell. The terms also encompass cells that have been modified by removing a nucleic acid endogenous to the cell by means of common well-known technologies for a skilled person (like, e.g., knocking-out genes).

A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied that will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell or microorganism as used within the context of the disclosure refers to a cell or microorganism that is genetically engineered.

The term “endogenous,” within the context of the disclosure refers to any polynucleotide, polypeptide or protein sequence that is a natural part of a cell and is occurring at its natural location in the cell chromosome. The term “exogenous” refers to any polynucleotide, polypeptide or protein sequence that originates from outside the cell under study and not a natural part of the cell or that is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified expression” of a gene relates to a change in expression compared to the wild-type expression of the gene in any phase of the production process of the desired sialylated di- and/or oligosaccharide. The modified expression is either a lower or higher expression compared to the wild type, wherein the term “higher expression” is also defined as “overexpression” of the gene in the case of an endogenous gene or “expression” in the case of a heterologous gene that is not present in the wild-type strain. Lower expression is obtained by means of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, . . . ), which are used to change the genes in such a way that they are less-able (i.e., statistically significantly “less able” compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can, for instance, be obtained by mutating one or more base pairs in the promoter sequence or changing the promoter sequence fully to a constitutive promoter with a lower expression strength compared to the wild type or an inducible promoter that results in regulated expression or a repressible promoter that results in regulated expression.

Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein the gene is part of an “expression cassette” that relates to any sequence in which a promoter sequence, untranslated region sequence (containing either a ribosome binding sequence, Shine Dalgarno or Kozak sequence), a coding sequence (for instance a sialyltransferase gene sequence) and optionally a transcription terminator is present, and leading to the expression of a functional active protein. The expression is either constitutive or conditional or regulated or tunable.

The term “constitutive expression” is defined as expression that is not regulated by transcription factors other than the subunits of RNA polymerase (e.g., the bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as a facultative or regulatory or tunable expression of a gene that is only expressed upon a certain natural condition of the host (e.g., mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminum, copper, zinc, nitrogen, phosphates, xylene, carbon or nitrogen depletion, or substrates or the produced product or chemical repression, as a response to an environmental change (e.g., anaerobic or aerobic growth, oxidative stress, pH shifts, temperature changes like, e.g., heat-shock or cold-shock, osmolarity, light conditions, starvation) or dependent on the position of the developmental stage or the cell cycle of the host cell including but not limited to apoptosis and autophagy. Regulated expression allows for control as to when a gene is expressed. The term “inducible expression by a natural inducer” is defined as a facultative or regulatory expression of a gene that is only expressed upon a certain natural condition of the host (e.g., organism being in labor, or during lactation), as a response to an environmental change (e.g., including but not limited to hormone, heat, cold, pH shifts, light, oxidative or osmotic stress/signaling), or dependent on the position of the developmental stage or the cell cycle of the host cell including but not limited to apoptosis and autophagy. The term “inducible expression upon chemical treatment” is defined as a facultative or regulatory expression of a gene that is only expressed upon treatment with a chemical inducer or repressor, wherein the inducer and repressor comprise but are not limited to an alcohol (e.g., ethanol, methanol), a carbohydrate (e.g., glucose, galactose, glycerol, lactose, arabinose, rhamnose, fucose, allo-lactose), metal ions (e.g., aluminum, copper, zinc), nitrogen, phosphates, IPTG, acetate, formate, xylene.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular host cell, cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

The term “modified expression of a protein” as used herein refers to i) higher expression or overexpression of an endogenous protein, ii) expression of a heterologous protein, iii) expression and/or overexpression of a variant protein that has a higher activity compared to the wild-type (i.e., native in the expression host) protein, iv) reduced expression of an endogenous protein or v) expression and/or overexpression of a variant protein that has a reduced activity compared to the wild-type (i.e., native in the expression host) protein. Preferably, the term “modified expression of a protein” as used herein refers to i) higher expression or overexpression of an endogenous protein, ii) expression of a heterologous protein or iii) expression and/or overexpression of a variant protein that has a higher activity compared to the wild-type (i.e., native in the expression host) protein.

The term “modified activity” of a protein relates to a non-native activity of the protein in any phase of the production process of the desired sialylated di- and/or oligosaccharide. The term “non-native,” as used herein with reference to the activity of a protein indicates that the protein has been modified to have an abolished, impaired, reduced, delayed, higher, accelerated or improved activity compared to the native activity of the protein. A modified activity of a protein is obtained by modified expression of the protein or is obtained by expression of a modified, i.e., mutant form of the protein, modified expression of the protein or is obtained by expression of a modified, i.e., mutant form of the protein. A mutant form of the protein can be obtained by expression of a mutant form of the gene encoding the protein, e.g., comprising a deletion, an insertion and/or a mutation of one or more nucleotides compared to the native gene sequence. A mutant form of a gene can be obtained by techniques well-known to a person skilled in the art, such as but not limited to site-specific mutation; CrispR; riboswitch; recombineering; ssDNA mutagenesis; transposon mutagenesis.

The term “non-native,” as used herein with reference to a cell producing a sialylated di- and/or oligosaccharide, indicates that the sialylated di- and/or oligosaccharide is i) not naturally produced or ii) when naturally produced not in the same amounts by the cell; and that the cell has been genetically modified to be able to produce the sialylated di- and/or oligosaccharide or to have a higher production of the sialylated di- and/or oligosaccharide.

As used herein, the term “mammary cell(s)” generally refers to mammalian mammary epithelial cell(s), mammalian mammary-epithelial luminal cell(s), or mammalian epithelial alveolar cell(s), or any combination thereof. As used herein, the term “mammary-like cell(s)” generally refers to mammalian cell(s) having a phenotype/genotype similar (or substantially similar) to natural mammalian mammary cell(s) but is/are derived from mammalian non-mammary cell source(s). Such mammalian mammary-like cell(s) may be engineered to remove at least one undesired genetic component and/or to include at least one predetermined genetic construct that is typical of a mammalian mammary cell. Non-limiting examples of mammalian mammary-like cell(s) may include mammalian mammary epithelial-like cell(s), mammalian mammary epithelial luminal-like cell(s), mammalian non-mammary cell(s) that exhibits one or more characteristics of a cell of a mammalian mammary cell lineage, or any combination thereof. Further non-limiting examples of mammalian mammary-like cell(s) may include mammalian cell(s) having a phenotype similar (or substantially similar) to natural mammalian mammary cell(s), or more particularly a phenotype similar (or substantially similar) to natural mammalian mammary epithelial cell(s). A mammalian cell with a phenotype or that exhibits at least one characteristic similar to (or substantially similar to) a natural mammalian mammary cell or a mammalian mammary epithelial cell may comprise a mammalian cell (e.g., derived from a mammary cell lineage or a non-mammary cell lineage) that exhibits either naturally, or has been engineered to, be capable of expressing at least one milk component.

As used herein, the term “non-mammary cell(s)” may generally include any mammalian cell of non-mammary lineage. In the context of the disclosure, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the disclosure contemplates making functional variants by modifying the structure of an enzyme as used in the disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide.

“Fragment,” with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO, typically, comprising or consisting of at least about 9, 10, 11, 12 consecutive nucleotides from the polynucleotide SEQ ID NO, for example, at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, “a fragment of a polynucleotide SEQ ID NO” preferably means a nucleotide sequence that comprises or consists of the polynucleotide SEQ ID NO wherein no more than about 200, 150, 100, 50 or 25 consecutive nucleotides are missing, preferably no more than about 50 consecutive nucleotides are missing, and that retains a usable, functional characteristic (e.g., activity) of the full-length polynucleotide molecule that can be assessed by the skilled person through routine experimentation. Alternatively, “a fragment of a polynucleotide SEQ ID NO” preferably means a nucleotide sequence that comprises or consists of an amount of consecutive nucleotides from the polynucleotide SEQ ID NO and wherein the amount of consecutive nucleotides is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 85.0%, even more preferably at least 87.0%, even more preferably at least 90.0%, even more preferably at least 95.0%, most preferably at least 97.0%, of the full-length of the polynucleotide SEQ ID NO and retains a usable, functional characteristic (e.g., activity) of the full-length polynucleotide molecule. As such, “a fragment of a polynucleotide SEQ ID NO” preferably means a nucleotide sequence that comprises or consists of the polynucleotide SEQ ID NO, wherein an amount of consecutive nucleotides is missing and wherein the amount is no more than 50.0%, 40.0%, 30.0% of the full-length of the polynucleotide SEQ ID NO (or GenBank NO.), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of the polynucleotide SEQ ID NO and wherein the fragment retains a usable, functional characteristic (e.g., activity) of the full-length polynucleotide molecule that can be routinely assessed by the skilled person.

“Fragment,” with respect to a polypeptide, refers to a subsequence of the polypeptide that performs at least one biological function of the intact polypeptide in substantially the same manner, or to a similar extent, as does the intact polypeptide. A “subsequence of the polypeptide” or “a stretch of amino acid residues” as defined herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example, at least about 10 amino acid residues in length, for example, at least about 20 amino acid residues in length, for example, at least about 30 amino acid residues in length, for example, at least about 100 amino acid residues in length, for example, at least about 150 amino acid residues in length, for example, at least about 200 amino acid residues in length. As such, “a fragment of a polypeptide SEQ ID NO (or UniProt ID)” preferably means a polypeptide sequence that comprises or consists of the polypeptide SEQ ID NO (or UniProt ID) wherein no more than about 200, 150, 125, 100, 80, 60, 50, 40, 30, 20 or 15 consecutive amino acid residues are missing, preferably no more than about 100 consecutive amino acid residues are missing, more preferably no more than about 50 consecutive amino acid residues are missing, even more preferably no more than about 40 consecutive amino acid residues are missing, and performs at least one biological function of the intact polypeptide in substantially the same manner, preferably to a similar or greater extent, as does the intact polypeptide that can be routinely assessed by the skilled person. Alternatively, “a fragment of a polypeptide SEQ ID NO (or UniProt ID)” preferably means a polypeptide sequence that comprises or consists of an amount of consecutive amino acid residues from the polypeptide SEQ ID NO (or UniProt ID) and wherein the amount of consecutive amino acid residues is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 85.0%, even more preferably at least 87.0%, even more preferably at least 90.0%, even more preferably at least 95.0%, most preferably at least 97.0% of the full-length of the polypeptide SEQ ID NO (or UniProt ID) and that performs at least one biological function of the intact polypeptide in substantially the same manner, preferably to a similar or greater extent, as does the intact polypeptide that can be routinely assessed by the skilled person. As such, “a fragment of a polypeptide SEQ ID NO (or UniProt ID)” preferably means a polypeptide sequence that comprises or consists of the polypeptide SEQ ID NO (or UniProt ID), wherein an amount of consecutive amino acid residues is missing and wherein the amount is no more than 50.0%, 40.0%, 30.0% of the full-length of the polypeptide SEQ ID NO (or UniProt ID or GenBank NO.), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of the polypeptide SEQ ID NO (or UniProt ID) and that performs at least one biological function of the intact polypeptide in substantially the same manner, preferably to a similar or greater extent, as does the intact polypeptide that can be routinely assessed by the skilled person.

Throughout the application, the sequence of a polypeptide can be represented by a SEQ ID NO or, alternatively, by a UniProt I. Therefore, the terms “polypeptide SEQ ID NO” and “polypeptide UniProt ID” and can be interchangeably used, unless explicitly stated otherwise.

A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions that have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describe those nucleotide sequences that have sequence similarity and encode polypeptides that share at least one functional characteristic such as a biochemical activity. More specifically, the term “functional homolog” as used herein describes those polypeptides that have sequence similarity and also share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLoS Comput. Biol. 8 (2012) e1002514).

Homologs can be identified by analysis of nucleotide and polypeptide sequence alignments. For example, performing a query on a database of nucleotide or polypeptide sequences can identify homologs of the polypeptide of interest. Sequence analysis can involve BLAST, Reciprocal BLAST, or PSI-BLAST analysis of non-redundant databases using amino acid sequence of a reference polypeptide sequence. The amino acid sequence is, in some instances, deduced from the nucleotide sequence. Typically, those polypeptides in the database that have greater than 40% sequence identity to a polypeptide of interest are candidates for further evaluation for suitability as a homologous polypeptide. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

A domain can be characterized, for example, by a Pfam (El-Gebali et al., Nucleic Acids Res. 47 (2019) D427-D432), an IPR (InterPro domain) (ebi.ac.uk/interpro) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (on the world-wide web at ncbi.nlm.nih.gov/cdd) (Lu et al., Nucleic Acids Res. 48 (2020) D265-D268), a PTHR domain (on the world-wide web at pantherdb.org) (Mi et al., Nucleic Acids. Res. 41 (2013) D377-D386; Thomas et al., Genome Research 13 (2003) 2129-2141) or a PATRIC identifier or PATRIC DB global family domain (on the world-wide web at patricbrc.org) (Davis et al., Nucleic Acids Res. 48 (D1) (2020) D606-D612). Protein or polypeptide sequence information and functional information can be provided by a comprehensive resource for protein sequence and annotation data like, e.g., the Universal Protein Resource (UniProt) (www.uniprot.org) (Nucleic Acids Res. 2021, 49 (D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. Throughout the application, the sequence of a polypeptide is represented by a SEQ ID NO or a UniProt ID. Unless stated otherwise, the UniProt IDs of the proteins described correspond to their sequence version 01 as present in the UniProt Database (www.uniprot.org) version release 2021_03 and consulted on 9 Jun. 2021. InterPro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool.

It should be understood for those skilled in the art that for the databases used herein, comprising Pfam 32.0 (released September 2018), CDD v3.17 (released 3 Apr. 2019), eggnogdb 4.5.1 (released September 2016), InterPro 75.0 (released 4 Jul. 2019), TCDB (released 17 Jun. 2019) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the % sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of a given SEQ ID NO, i.e., the reference sequence, resulting in a global % identity score. Alternatively, % identity may be calculated over a partial sequence of the reference sequence, resulting in a local percent identity score. A “partial sequence” preferably means at least about 25%, 30%, 35%, 40%, 45%, 50%, 55%, 60%, 65%, 70%, 75%, 80%, 85%, 87.5%, 90%, 91%, 92%, 93%, 94% or 95% of the full-length reference sequence. In another preferred embodiment, “a partial sequence of a reference polypeptide sequence” means a stretch of at least 150 amino acid residues up to the total number of amino acid residues of a reference polypeptide sequence. In another more preferred embodiment, “a partial sequence of a reference polypeptide sequence” means a stretch of at least 200 amino acid residues up to the total number of amino acid residues of a reference polypeptide sequence. Using the full-length of the reference sequence in a local sequence alignment results in a global percent identity score between the test and the reference sequence.

Percent identity can be determined using different algorithms like, for example, BLAST and PSI-BLAST (Altschul et al., 1990, J. Mol. Biol. 215:3, 403-410; Altschul et al., 1997, Nucleic Acids Res. 25:17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

As used herein, a polypeptide comprising or consisting of an amino acid sequence having 80.0% or more sequence identity over a stretch of at least 200 amino acid residues of a reference polypeptide sequence is to be understood as that the amino acid sequence has 80.0%, 81.0%, 82.0%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity over a stretch of at least 200 amino acid residues of the reference polypeptide sequence.

As used herein, a polypeptide comprising or consisting of an amino acid sequence having 80.0% or more sequence identity to the full-length sequence of a reference polypeptide sequence is to be understood as that the amino acid sequence has 80.0%, 81.0%, 82.0%, 83.0%, 84.0%, 85.0%, 86.0%, 87.0%, 88.0%, 89.0%, 90.0%, 91.0%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of the amino acid sequence of the reference polypeptide sequence.

Throughout the application, unless explicitly specified otherwise, a polypeptide comprising, consisting of or having an amino acid sequence having 80.0% or more sequence identity to the full-length amino acid sequence of a reference polypeptide, usually indicated with a SEQ ID NO or UniProt ID, preferably has 80.0%, 85.0%, 90.0%, 91.0%, 92.0%, 93.0%, 94.0%, 95.0%, 96.0%, 97.0%, 98.0% or 99.0%, more preferably has at least 85.0%, even more preferably has at least 87.50%, even more preferably has at least 90.0%, sequence identity to the full length reference sequence. Additionally, unless explicitly specified otherwise, a polynucleotide sequence comprising, consisting of or having a nucleotide sequence having 80.0% or more sequence identity to the full-length nucleotide sequence of a reference polynucleotide sequence, usually indicated with a SEQ ID NO, preferably has 80.0%, 85.0%, 90.0%, 91.0%, 92.0%, 93.0%, 94.0%, 95.0%, 96.0%, 97.0%, 98.0% or 99.0%, more preferably has at least 85.0%, even more preferably has at least 87.50%, even more preferably has at least 90.0% sequence identity to the full-length reference polynucleotide sequence.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

The terms “sialic acid,” “Neu5Ac,” “N-acetylneuraminate,” “N-acylneuraminate,” “N-acetylneuraminic acid” are used interchangeably and refer to an acidic sugar with a nine-carbon backbone.

Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C11H19NO9 as molecular formula.

The terms “Neu5Ac synthase,” “N-acetylneuraminic acid synthase,” “N-acetylneuraminate synthase,” “sialic acid synthase,” “NeuAc synthase,” “NeuB,” “NeuB1,” “NANA condensing enzyme,” “N-acetylneuraminate lyase synthase,” “N-acetylneuraminic acid condensing enzyme” as used herein are used interchangeably and refer to an enzyme capable to synthesize sialic acid (Neu5Ac) from N-acetylmannosamine (ManNAc) in a reaction using phosphoenolpyruvate (PEP).

The terms “CMP-sialic acid synthase,” “N-acylneuraminate cytidylyltransferase,” “CMP-sialate synthase,” “CMP-Neu5Ac synthase,” “CMP-NeuAc synthase,” “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “N-acylneuraminate-9-phosphate synthetase,” “NANA synthase,” “NANAS,” “NANS,” “NmeNANAS,” “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesize N-acylneuraminate.

An N-acylglucosamine 2-epimerase is an enzyme that catalyzes the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase, N-acetylglucosamine epimerase and N-acyl-D-glucosamine 2-epimerase.

A UDP-N-acetylglucosamine 2-epimerase is an enzyme that catalyzes the reaction N-acetyl-D-glucosamine=N-acetylmannosamine. Alternative names for this enzyme comprise UDP-N-acylglucosamine 2-epimerase, UDP-GlcNAc-2-epimerase, neuC and UDP-N-acetyl-D-glucosamine 2-epimerase.

An N-acetylmannosamine-6-phosphate 2-epimerase is an enzyme that catalyzes the reaction N-acetyl-D-glucosamine 6-phosphate=N-acetyl-D-mannosamine 6-phosphate.

A bifunctional UDP-GlcNAc 2-epimerase/kinase is a bifunctional enzyme that catalyzes the reaction UDP-N-acetyl-D-glucosamine=N-acetyl-D-mannosamine and the reaction N-acetyl-D-mannosamine+ATP=ADP+N-acetyl-D-mannosamine 6-phosphate.

The terms “N-acetylneuraminate lyase,” “Neu5Ac lyase,” “N-acetylneuraminate pyruvate-lyase,” “N-acetylneuraminic acid aldolase,” “NALase,” “sialate lyase,” “sialic acid aldolase,” “sialic acid lyase” and “nanA” are used interchangeably and refer to an enzyme that degrades N-acetylneuraminate into N-acetylmannosamine (ManNAc) and pyruvate.

The terms “N-acetylneuraminate kinase,” “ManNAc kinase,” “N-acetyl-D-mannosamine kinase” and “nanK” are used interchangeably and refer to an enzyme that phosphorylates ManNAc to synthesize N-acetylmannosamine-phosphate (ManNAc-6-P).

The terms “N-acetylneuraminate epimerase,” “ManNAc-6-P isomerase,” “ManNAc-6-P 2-epimerase” and “nanE” are used interchangeably and refer to an enzyme that catalyzes the reaction N-acetyl-D-glucosamine 6-phosphate=N-acetyl-D-mannosamine 6-phosphate.

A glucosamine 6-phosphate N-acetyltransferase is an enzyme that catalyzes the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosamine-phosphate N-acetyltransferase, glucosamine-6-phosphate acetylase, N-acetylglucosamine-6-phosphate synthase, phosphoglucosamine acetylase, phosphoglucosamine N-acetylase phosphoglucosamine N-acetylase, phosphoglucosamine transacetylase, GNA and GNA1.

The term “N-acetylglucosamine-6-phosphate phosphatase” refers to an enzyme that dephosphorylates N-acetylglucosamine-6-phosphate (GlcNAc-6-P) hereby synthesizing N-acetylglucosamine (GlcNAc).

The term “N-acetylmannosamine-6-phosphate phosphatase” refers to an enzyme that dephosphorylates N-acetylmannosamine-6-phosphate (ManNAc-6P) to N-acetylmannosamine (ManNAc).

The terms “N-acetylglucosamine-6-P deacetylase,” “N-acetylglucosamine-6-phosphate deacetylase” and “nagA” are used interchangeably and refer to an enzyme that catalyzes the hydrolysis of the N-acetyl group of N-acetylglucosamine-6-phosphate (GlcNAc-6-P) to yield glucosamine-6-phosphate (GlcN6P) and acetate.

The terms “glucosamine-6-P deaminase,” “glucosamine-6-phosphate deaminase,” “GlcN6P deaminase,” “glucosamine-6-phosphate isomerase,” “glmD” and “nagB” are used interchangeably and refer to an enzyme that catalyzes the reversible isomerization-deamination of glucosamine-6-phosphate (GlcN6P) to form fructose-6-phosphate and an ammonium ion.

The terms “L-glutamine-D-fructose-6-phosphate aminotransferase,” “glutamine-fructose-6-phosphate transaminase (isomerizing),” “hexosephosphate aminotransferase,” “glucosamine-6-phosphate isomerase (glutamine-forming),” “glutamine-fructose-6-phosphate transaminase (isomerizing),” “D-fructose-6-phosphate amidotransferase,” “fructose-6-phosphate aminotransferase,” “glucosaminephosphate isomerase,” “glucosamine 6-phosphate synthase,” “GlcN6P synthase,” “GFA,” “glms,” “glmS” and “glmS*54” are used interchangeably and refer to an enzyme that catalyzes the conversion of D-fructose-6-phosphate into D-glucosamine-6-phosphate using L-glutamine.

The terms “phosphoglucosamine mutase” and “glmM” are used interchangeably and refer to an enzyme that catalyzes the conversion of glucosamine-6-phosphate to glucosamine-1-phosphate. Phosphoglucosamine mutase can also catalyze the formation of glucose-6-P from glucose-1-P, although at a 1400-fold lower rate.

The terms “phosphoacetylglucosamine mutase,” “acetylglucosamine phosphomutase,” “acetylaminodeoxyglucose phosphomutase,” “phospho-N-acetylglucosamine mutase” and “N-acetyl-D-glucosamine 1,6-phosphomutase” are used interchangeably and refer to an enzyme that catalyzes the conversion of N-acetyl-glucosamine 1-phosphate into N-acetylglucosamine 6-phosphate.

The terms “N-acetylglucosamine 1-phosphate uridylyltransferase,” “N-acetylglucosamine-1-phosphate uridyltransferase,” “UDP-N-acetylglucosamine diphosphorylase,” “UDP-N-acetylglucosamine pyrophosphorylase,” “uridine diphosphoacetylglucosamine pyrophosphorylase,” “UTP: 2-acetamido-2-deoxy-alpha-D-glucose-1-phosphate uridylyltransferase,” “UDP-GlcNAc pyrophosphorylase,” “GlmU uridylyltransferase,” “Acetylglucosamine 1-phosphate uridylyltransferase,” “UDP-acetylglucosamine pyrophosphorylase,” “uridine diphosphate-N-acetylglucosamine pyrophosphorylase,” “uridine diphosphoacetylglucosamine phosphorylase,” and “acetylglucosamine 1-phosphate uridylyltransferase” are used interchangeably and refer to an enzyme that catalyzes the conversion of N-acetylglucosamine 1-phosphate (GlcNAc-1-P) into UDP-N-acetylglucosamine (UDP-GlcNAc) by the transfer of uridine 5-monophosphate (from uridine 5-triphosphate (UTP)).

The term “glucosamine-1-phosphate acetyltransferase” refers to an enzyme that catalyzes the transfer of the acetyl group from acetyl coenzyme A to glucosamine-1-phosphate (GlcN-1-P) to produce N-acetylglucosamine-1-phosphate (GlcNAc-1-P).

The term “glmU” refers to a bifunctional enzyme that has both N-acetylglucosamine-1-phosphate uridyltransferase and glucosamine-1-phosphate acetyltransferase activity and that catalyzes two sequential reactions in the de novo biosynthetic pathway for UDP-GlcNAc. The C-terminal domain catalyzes the transfer of acetyl group from acetyl coenzyme A to GlcN-1-P to produce GlcNAc-1-P, which is converted into UDP-GlcNAc by the transfer of uridine 5-monophosphate, a reaction catalyzed by the N-terminal domain.

The term “glycosyltransferase” as used herein refers to an enzyme capable to catalyze the transfer of sugar moieties from activated donors to specific acceptors, forming glycosidic bonds. The donor can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, erythritol, Arabinitol, xylitol, ribitol, glucitol, galactitol, mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulo-pyranosonic acid, 2-acetamido-2,6-dideoxy--L-arabino-4-hexulose, 2-acetamido-2,6-dideoxy--L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Galn), fucose (Fuc), rhamnose (Rha), glucuronic acid, gluconic acid, fructose (Fru) and polyols. With the term “polyol” is meant an alcohol containing multiple hydroxyl groups. For example, glycerol, sorbitol, or mannitol.

The term “phosphorylated monosaccharide” as used herein refers to one of the above-listed monosaccharides, which is phosphorylated. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The terms “activated monosaccharide,” “nucleotide-activated sugar,” “nucleotide-sugar,” “activated sugar,” “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), GDP-fucose (GDP-Fuc), GDP-rhamnose and UDP-xylose. Nucleotide-sugars act as glycosyl donors in glycosylation reactions. Glycosylation reactions are reactions that are catalyzed by glycosyltransferases.

The term “disaccharide” as used herein refers to a saccharide polymer containing two simple sugars, i.e., monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharide” as the term is used herein and as generally understood in the state of the art, refers to a saccharide polymer containing a small number, typically three to twenty, preferably three to ten, of simple sugars, i.e., monosaccharides. Preferably, the oligosaccharide as described herein contains monosaccharides selected from the list as used herein above. The oligosaccharide as used in the disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc,” “Gal-b1,4-Glc,” “b-Gal-(1->4)-Glc,” “b-Gal-(1->4)-Glc,” “Galbeta 1-4-Glc,” “Gal-b (1-4)-Glc” and “Gal-b (1-4)-Glc” have the same meaning, i.e., a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars, antigens of the human ABO blood group system, a neutral (non-charged) oligosaccharide, a negatively charged oligosaccharide, a fucosylated oligosaccharide, a sialylated oligosaccharide, an N-acetylglucosamine containing oligosaccharide, an N-acetyllactosamine containing oligosaccharide, a lacto-N-biose containing oligosaccharide, a lactose containing oligosaccharide, a non-fucosylated neutral (non-charged) oligosaccharide, an N-acetyllactosamine containing fucosylated oligosaccharide, an N-acetyllactosamine non-fucosylated oligosaccharide, a lacto-N-biose containing fucosylated oligosaccharide, a lacto-N-biose containing non-fucosylated oligosaccharide, an N-acetyllactosamine containing negatively charged oligosaccharide, a lacto-N-biose containing negatively charged oligosaccharide, an animal oligosaccharide, preferably selected from the group consisting of N-glycans and O-glycans, a plant oligosaccharide, preferably selected from the group consisting of N-glycans and O-glycans.

As used herein, a “sialylated oligosaccharide” is to be understood as a charged sialic acid containing oligosaccharide, i.e., an oligosaccharide having a sialic acid residue. It has an acidic nature. Some examples are 3-SL (3′-sialyllactose or 3′SL or Neu5Ac-a2,3-Gal-b1,4-Glc), 3′-sialyllactosamine, 6-SL (6′sialyllactose, 6′-sialyllactose or 6′SL or Neu5 Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′sialyllactose (also known as 6′sialyllactose, 6′SL and 6′-SL), SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3) Galβ-1,4Glc, GM1 Galβ-1,3GalNAcβ-1,4 (Neu5Acα-2,3) Galβ-1,4Glc, GD1a Neu5Acα-2,3Galβ-1,3GalNAcβ-1,4 (Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3) Galβ-1,4Glc, GT2 GalNAcβ-1,4 (Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3) Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3) Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAcβ-1,4 (Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

As used herein, a “6′sialylated oligosaccharide” is to be understood as a charged sialic acid containing oligosaccharide comprising an oligosaccharide or disaccharide, which is alpha-2,6-glycosidically linked to a sialic acid residue.

A “neutral oligosaccharide” or a “non-charged oligosaccharide” as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′,3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

Mammalian milk oligosaccharides comprise oligosaccharides present in milk found in any phase during lactation including colostrum milk from humans and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus). As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides. Human milk oligosaccharides are also known as human identical milk oligosaccharides that are chemically identical to the human milk oligosaccharides found in human breast milk, but that are biotechnologically produced (e.g., using cell free systems or cells and organisms comprising a bacterium, a fungus, a yeast, a plant, animal, or protozoan cell, preferably metabolically engineered cells and organisms). Human identical milk oligosaccharides are marketed under the name HiMO. HMOs comprise fucosylated oligosaccharides, non-fucosylated neutral oligosaccharides and sialylated oligosaccharides (see, e.g., Chen X., Chapter Four: Human Milk Oligosaccharides (HMOS): Structure, Function, and Enzyme-Catalyzed Synthesis in Adv. Carbohydr. Chem. Biochem. 72, 113 (2015)). Examples of HMOs comprise 3-fucosyllactose, 2′-fucosyllactose, 2′,3-difucosyllactose, 6′-sialyllactose, 3′-sialyllactose, LN3, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, difucosyllacto-N-tetraose, lacto-N-hexaose, lacto-N-difucohexaose I, lacto-N-difucohexaose II, disialyllacto-N-tetraose, fucosyllacto-N-hexaose, difucosyllacto-N-hexaose, fucodisialyllacto-N-hexaose, disialyllacto-N-hexaose. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. The structures involve the A determinant GalNAc-alpha1,3 (Fuc-alpha1,2)-Gal-, the B determinant Gal-alpha1,3 (Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4Glc” as used in the disclosure, are used interchangeably.

The terms “LNT,” “lacto-N-tetraose,” “lacto-N-tetraose” or “Galβ1-3GlcNAcβ1-3Galβ1-4Glc” as used in the present disclosure, are used interchangeably.

The terms “LNnT,” “lacto-N-neotetraose,” “lacto-N-neotetraose,” “neo-LNT” or “Galβ1-4GlcNAcβ1-3Galβ1-4Glc” as used in the disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in the disclosure, are used interchangeably.

The terms “LSTb,” “LS-Tetrasaccharide b,” “Sialyl-lacto-N-tetraose b,” “sialyllacto-N-tetraose b” or “Gal-b1,3-(Neu5Ac-a2,6)-GlcNAc-b1,3-Gal-b1,4-Glc” as used in the disclosure, are used interchangeably.

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in the disclosure, are used interchangeably.

The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc” as used in the disclosure, are used interchangeably.

The term “pathway for production of a sialylated di and/or oligosaccharide” as used herein is a biochemical pathway consisting of the enzymes and their respective genes involved in the synthesis of a sialylated di- and/or oligosaccharide as defined herein. The pathway for production of a sialylated di- and/or oligosaccharide comprises at least one Neu5Ac synthase. Furthermore, the pathway for production of a sialylated di- and/or oligosaccharide can comprise but is not limited to pathways involved in the synthesis of a nucleotide-activated sugar and the transfer of the nucleotide-activated sugar to an acceptor to create a sialylated di- and/or oligosaccharide of the disclosure. Further examples of such pathway comprise but are not limited to a fucosylation, sialylation, galactosylation, N-acetylglucosaminylation, N-acetylgalactosylation, mannosylation, N-acetylmannosaminylation pathway.

A “sialylation pathway” is a biochemical pathway comprising at least one of the enzymes and their respective genes chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthetase, N-acylneuraminate-9-phosphate phosphatase, Neu5Ac synthase and CMP sialic acid synthase combined with a sialyltransferase leading to α 2,3; α 2,6 and/or α 2,8 sialylated compounds.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50% 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy. Further herein, the terms “contaminants” and “impurities” preferably mean particulates, cells, cell components, metabolites, cell debris, proteins, peptides, amino acids, nucleic acids, glycolipids and/or endotoxins that can be present in an aqueous medium like, e.g., a cultivation or an incubation.

The term “clarifying” as used herein refers to the act of treating an aqueous medium like, e.g., a cultivation or an incubation, to remove suspended particulates and contaminants from the production process like, e.g., cells, cell components, insoluble metabolites and debris, that could interfere with the eventual purification of the one or more bioproduct(s). Such treatment can be carried out in a conventional manner by centrifugation, flocculation, flocculation with optional ultrasonic treatment, gravity filtration, microfiltration, foam separation or vacuum filtration (e.g., through a ceramic filter that can include a Celite™ filter aid).

The terms “cultivation” and “incubation” are used interchangeably and refer to the culture medium wherein the cell is cultivated, incubated or fermented, the cell itself, and the 6′sialylated disaccharide and/or 6′sialylated oligosaccharide, preferably 6′sialyllactose, that is produced by the cell in whole broth, i.e., inside (intracellularly) as well as outside (extracellularly) of the cell.

As used herein, the term “cell productivity index (CPI)” refers to the mass of the sialylated di- and/or oligosaccharide produced by the cells divided by the mass of the cells produced in the culture.

The term “precursor” as used herein refers to substances that are taken up or synthetized by the cell for the specific production of a sialylated di- and/or oligosaccharide according to the disclosure. In this sense, a precursor can be an acceptor as defined herein, but can also be another substance, metabolite, which is first modified within the cell as part of the biochemical synthesis route of the sialylated di- and/or oligosaccharide. The term “precursor” as used herein is also to be understood as a chemical compound that participates in a chemical or enzymatic reaction to produce another compound like, e.g., an intermediate, as part in the metabolic pathway of a sialylated di- and/or oligosaccharide according to the disclosure. The term “precursor” as used herein is also to be understood as a donor that is used by a glycosyltransferase to modify an acceptor substrate with a sugar moiety in a glycosidic bond, as part in the metabolic pathway of a sialylated di- and/or oligosaccharide according to the disclosure. Examples of such precursors comprise the acceptors as defined herein, and/or glucose, galactose, fructose, glycerol, sialic acid, fucose, mannose, maltose, sucrose, lactose, glucose-1-phosphate, galactose-1-phosphate, UDP-glucose, UDP-galactose, glucose-6-phosphate, fructose-6-phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetyl-glucosamine-6-phosphate, N-acetyl-glucosamine, N-acetyl-mannosamine, N-acetylmannosamine-6-phosphate, UDP-N-acetylglucosamine, N-acetylglucosamine-1-phosphate, N-acetyl-Neuraminic acid-9-phosphate, CMP-sialic acid, mannose-6-phosphate, mannose-1-phosphate, GDP-mannose, GDP-4-dehydro-6-deoxy-α-D-mannose, and/or GDP-fucose.

Optionally, the cell is transformed to comprise and to express at least one nucleic acid sequence encoding a protein selected from the group consisting of lactose transporter, N-acetylneuraminic acid transporter, fucose transporter, glucose transporter, galactose transporter, transporter for a nucleotide-activated sugar wherein the transporter internalizes a to the medium added precursor for the synthesis of the sialylated di- and/or oligosaccharide, preferably 6′sialyllactose, of the disclosure.

The term “acceptor” as used herein refers to a mono-, di- or oligosaccharide, which can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with beta-GlcNAc-Asn residues, glycoproteins with terminal GlcNAc and Gal residues, immunoglobulins.

### DETAILED DESCRIPTION

According to a first embodiment, the disclosure provides an alpha-2,6-sialyltransferase for use in the production of a 6′sialylated disaccharide and/or 6′sialylated oligosaccharide, preferably 6′sialyllactose, wherein the alpha-2,6-sialyltransferase comprises an amino acid sequence that is at least to 80.0% identical over a stretch of at least 200 amino acids to the amino acid sequence as represented by SEQ ID NO: 1, preferably the alpha-2,6-sialyltransferase is a lactose-accepting alpha-2,6-sialyltransferase.

Preferably, the alpha-2,6-sialyltransferase comprises an amino acid sequence that is at least 80.0%, at least 85.0%, at least 90.0%, at least 95.0%, at least 96.0%, at least 97.0%, at least 98.0%, at least 98.5%, or at least 99% identical to the amino acid sequence as represented by SEQ ID NO: 1 over a stretch of at least 210, at least 220, at least 230, at least 240, at least 250, at least 260, at least 270, at least 280, at least 290 or at least 300 amino acids and comprising alpha-2,6-sialyltransferase activity. More preferably, the alpha-2,6-sialyltransferase comprises an amino acid sequence that is at least 80.0%, at least 85.0%, at least 90.0%, at least 95.0%, at least 96.0%, at least 97.0%, at least 98.0%, at least 98.5%, or at least 99% identical to the amino acid sequence as represented by SEQ ID NO: 1 and comprising alpha-2,6-sialyltransferase activity. Alternatively, the alpha-2,6-sialyltransferase comprises an amino acid sequence comprising a functional fragment of an amino acid sequence as represented by SEQ ID NO: 1. Preferably, the alpha-2,6-sialyltransferase is a lactose-accepting alpha-2,6-sialyltransferase, thus having a lactose-accepting alpha-2,6-sialyltransferase activity. Most preferably, the alpha-2,6-sialyltransferase comprises an amino acid sequence as represented by SEQ ID NO: 1.

In a second embodiment, the disclosure provides a method for the production of a 6′sialylated disaccharide and/or 6′sialylated oligosaccharide, preferably 6′sialyllactose. The method comprising contacting the isolated sialyltransferase as described herein with a mixture comprising a donor substrate comprising a sialic acid residue, and an acceptor substrate comprising an oligosaccharide or disaccharide, under conditions wherein the sialyltransferase catalyzes the transfer of a sialic acid residue from the donor substrate to the acceptor substrate, thereby producing the 6′sialylated disaccharide and/or 6′sialylated oligosaccharide. Preferably, the 6′sialylated disaccharide and/or 6′sialylated oligosaccharide is a 6′sialyllactose and the acceptor substrate is the disaccharide lactose.

In a specific embodiment, the disclosure provides a method for the production of a 6′sialylated disaccharide and/or 6′sialylated oligosaccharide, preferably 6′sialyllactose, wherein the method comprises the steps of:
a) Providing
   i. CMP-sialic acid
   ii. An acceptor substrate, preferably lactose
   iii. A sialyltransferase, wherein the sialyltransferase is an alpha-2,6-sialyltransferase that comprises an amino acid sequence that is at least to 80.0% identical over a stretch of at least 200 amino acids to the amino acid sequence as represented by SEQ ID NO: 1,
b) contacting the sialyltransferase and CMP-sialic acid with the acceptor substrate, under conditions where the sialyltransferase catalyzes the transfer of a sialic acid residue from the CMP-sialic acid to the acceptor substrate resulting in the production of a 6′sialylated disaccharide and/or 6′sialylated oligosaccharide, preferably 6′sialyllactose,
c) preferably, separating the produced 6′sialylated disaccharide and/or 6′sialylated oligosaccharide, preferably a 6′sialyllactose, more preferably 6′sialyllactose.

Preferably, the sialyltransferase is any one of the alpha-2,6-sialyltransferase as described herein.

In an alternative specific embodiment, the disclosure provides a method wherein a cell extract comprising the sialyltransferase as described herein is contacted with a mixture comprising a donor substrate comprising a sialic acid residue, and an acceptor substrate comprising an oligosaccharide or disaccharide, under conditions where the sialyltransferase catalyzes the transfer of a sialic acid residue from the donor substrate to the acceptor substrate, thereby producing the sialylated di- and/or oligosaccharide. Preferably, the 6′sialylated disaccharide and/or 6′sialylated oligosaccharide is a 6′sialyllactose.

In an alternative embodiment of the method of the disclosure, the 6′sialylated disaccharide and/or 6′sialylated oligosaccharide is produced in a cell-free system. Preferably, the 6′sialylated disaccharide and/or 6′sialylated oligosaccharide is a 6′sialyllactose.

In a further alternative embodiment of the method of the disclosure, the 6′sialylated disaccharide and/or 6′sialylated oligosaccharide is produced by a cell, preferably a single cell, wherein the cell expresses an alpha-2,6-sialyltransferase that comprises an amino acid sequence that is at least to 80.0% identical over a stretch of at least 200 amino acids to the amino acid sequence as represented by SEQ ID NO: 1 or any of the alpha-2,6-sialyltransferases as described herein.

The cell used can be a metabolically engineered cell as described herein, preferably wherein the cell is metabolically engineered for the production of the 6′sialylated disaccharide and/or 6′sialylated oligosaccharide.

A further method according to the disclosure provides a method comprising the steps of:
i. providing a cell expressing, preferably heterologously expressing, more preferably heterologously overexpressing, the sialyltransferase as described herein,
ii. providing CMP-sialic acid, optionally, the CMP-sialic acid is produced by the cell, and
iii. providing an oligosaccharide or disaccharide, optionally, the oligosaccharide or disaccharide is produced by the cell, and
iv. cultivating and/or incubating the cell under conditions permissive to express the sialyltransferase, optionally permissive to produce the CMP-sialic acid and/or the oligosaccharide or disaccharide,
v. preferably, separating the 6′sialylated disaccharide and/or 6′sialylated oligosaccharide from the cultivation.

In the scope of the disclosure, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 7+/−3.

Preferably, the 6′sialylated disaccharide and/or 6′sialylated oligosaccharide is a 6′sialyllactose and the disaccharide is the disaccharide lactose.

In an embodiment of the disclosure, the cultivation or incubating medium contains at least one carbon source selected from the group consisting of glucose, fructose, sucrose, and glycerol. Preferably or alternatively, the cultivation or incubating medium contains at least one compound selected from the group consisting of lactose, galactose and sialic acid.

Preferably, the 6′sialylated disaccharide and/or 6′sialylated oligosaccharide is recovered from the cultivation or incubating medium and/or the cell; or separated from the cultivation as explained herein.

In a further preferred aspect, the method for the production of a 6′sialylated disaccharide and/or 6′sialylated oligosaccharide as described herein comprises at least one of the following steps:
i) Adding to the culture medium in a reactor at least one precursor and/or acceptor feed wherein the total reactor volume ranges from 250 mL (milliliter) to 10,000 m³ (cubic meter), preferably in a continuous manner, and preferably so that the final volume of the culture medium is not more than three-fold, preferably not more than two-fold, more preferably less than two-fold of the volume of the culture medium before the addition of the precursor and/or acceptor feed;
ii) Adding at least one precursor and/or acceptor feed in a continuous manner to the culture medium over the course of 1 day, 2 days, 3 days, 4 days, 5 days by means of a feeding solution;
iii) Adding at least one precursor and/or acceptor feed in a continuous manner to the culture medium over the course of 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
   the method resulting in a 6′sialylated disaccharide and/or 6′sialylated oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

In another and/or additional further preferred aspect, the method for the production of a 6′sialylated disaccharide and/or 6′sialylated oligosaccharide as described herein comprises at least one of the following steps:
i) Adding to the culture medium at least one precursor and/or acceptor in one pulse or in a discontinuous (pulsed) manner wherein the total reactor volume ranges from 250 mL (milliliter) to 10,000 m³ (cubic meter), preferably so that the final volume of the culture medium is not more than three-fold, preferably not more than two-fold, more preferably less than two-fold of the volume of the culture medium before the addition of the precursor and/or acceptor feed pulse(s);
ii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
iii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
   the method resulting in a 6′sialylated disaccharide and/or 6′sialylated oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

In a further, more preferred aspect, the method for the production of a 6′sialyllactose as described herein comprises at least one of the following steps:
i) Adding to the culture medium a lactose feed comprising at least 50, more preferably at least 75, more preferably at least 100, more preferably at least 120, more preferably at least 150 gram of lactose per liter of initial reactor volume wherein the total reactor volume ranges from 250 mL (milliliter) to 10,000 m³ (cubic meter), preferably in a continuous manner, and preferably so that the final volume of the culture medium is not more than three-fold, preferably not more than two-fold, more preferably less than two-fold of the volume of the culture medium before the addition of the lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7; and
wherein preferably the temperature of the feed solution is kept between 20° C. and 80° C.;
   the method resulting in 6′sialyllactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

Preferably the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.

In another aspect the lactose feed is accomplished by adding lactose to the cultivation medium in a concentration, such that throughout the production phase of the cultivation a lactose concentration of at least 5 mM, preferably 10 mM or 30 mM is obtained.

In a further embodiment of the methods described herein the cells are cultivated for at least about 60, 80, 100, or about 120 hours or in a continuous manner.

In a preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per liter of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon source, preferably glucose or sucrose, to the culture medium before the lactose is added to the culture medium in a second phase.

In an alternative preferable embodiment, in the method as described herein, the lactose is added already in the first phase of exponential growth together with the carbon-based substrate.

According to a third embodiment, the disclosure provides a metabolically engineered cell for the production of a 6′sialylated disaccharide and/or 6′sialylated oligosaccharide, preferably 6′sialyllactose, wherein the cell has been metabolically engineered to possess an alpha-2,6-sialyltransferase that comprises an amino acid sequence that is at least to 80.0% identical over a stretch of at least 200 amino acids to the amino acid sequence as represented by SEQ ID NO: 1, preferably, the alpha-2,6-sialyltransferase is a lactose-accepting alpha-2,6-sialyltransferase. Preferably, the alpha-2,6-sialyltransferase used in the cell is an alpha-2,6-sialyltransferase as described herein.

Alternatively or preferably, the cell contains a nucleic acid molecule that comprises a nucleotide sequence that encodes any one of the alpha-2,6-sialyltransferases as described herein.

Herein, a metabolically engineered cell comprising a pathway for production of the sialylated di- and/or oligosaccharide is provided. Examples of such pathways comprise but are not limited to pathways involved in the synthesis of monosaccharide, phosphorylated monosaccharide, nucleotide-activated sugar, and/or glycosylation pathways like, e.g., a fucosylation, sialylation, galactosylation, N-acetylglucosaminylation, N-acetylgalactosylation, mannosylation and/or N-acetylmannosaminylation pathway. The pathway for production of a sialylated di- and/or oligosaccharide preferably comprises at least one alpha-2,6-sialyltransferase as described herein.

In a preferred embodiment of the method and/or cell of the disclosure, the cell comprises one or more pathway(s) for monosaccharide synthesis. The pathways for monosaccharide synthesis comprise enzymes like, e.g., carboxylases, decarboxylases, isomerases, epimerases, reductases, enolases, phosphorylases, carboxykinases, kinases, phosphatases, aldolases, hydrolases, dehydrogenases, enzymes involved in the synthesis of one or more nucleoside triphosphate(s) like UTP, GTP, ATP and CTP, enzymes involved in the synthesis of any one or more nucleoside mono- or diphosphates like, e.g., UMP and UDP, respectively, and enzymes involved in the synthesis of phosphoenolpyruvate (PEP).

In another and/or additional preferred embodiment of the method and/or cell of the disclosure, the cell comprises one or more pathway(s) for phosphorylated monosaccharide synthesis. The pathways for phosphorylated monosaccharide synthesis comprise enzymes involved in the synthesis of one or more monosaccharide(s), one or more nucleoside mono-, di- and/or triphosphate(s) and enzymes involved in the synthesis of phosphoenolpyruvate (PEP) like, e.g., but not limited to PEP synthase, carboxylases, decarboxylases, isomerases, epimerases, reductases, enolases, phosphorylases, carboxykinases, kinases, phosphatases, aldolases, hydrolases and dehydrogenases. In another and/or additional preferred embodiment of the method and/or cell of the disclosure, the cell comprises one or more pathways for the synthesis of one or more nucleotide-activated sugars. The pathways for nucleotide-activated sugar synthesis comprise enzymes like, e.g., PEP synthase, carboxylases, decarboxylases, isomerases, epimerases, reductases, enolases, phosphorylases, carboxykinases, kinases, phosphatases, aldolases, hydrolases, dehydrogenases, mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, glucophosphomutase and/or N-acetylglucosamine-1-phosphate uridylyltransferase.

The cell may further comprise and express at least one further glycosyltransferase that is involved in the production of the 6′sialylated disaccharide and/or 6′sialylated oligosaccharide.

In a preferred embodiment of the method and/or cell of the disclosure, the cell is metabolically engineered to comprise a pathway for production of a 6′sialylated disaccharide and/or 6′sialylated oligosaccharide, preferably 6′sialyllactose, as defined herein. In an alternative preferred embodiment of the method and/or cell of the disclosure, the cell is metabolically engineered to comprise a pathway for production of a sialylated di- and/or oligosaccharide as defined herein, preferably for production of a 6′sialylated disaccharide and/or 6′sialylated oligosaccharide, preferably 6′sialyllactose, and to have modified expression or activity of an alpha-2,6-sialyltransferase of the disclosure.

In a further preferred embodiment of the method and/or cell of the disclosure, the cell comprises a recombinant alpha-2,6-sialyltransferase capable of modifying lactose or another acceptor as defined herein with one or more sialic acid molecules that is/are synthesized by any one or more Neu5Ac synthases expressed in the cell, into a 6′sialylated disaccharide and/or 6′sialylated oligosaccharide, preferably 6′sialyllactose.

In a preferred aspect of the method and/or cell of the disclosure, the metabolically engineered cell is modified with gene expression modules wherein the expression from any one of the expression modules is constitutive or is tunable.

The expression modules are also known as transcriptional units and comprise polynucleotides for expression of recombinant genes including coding gene sequences and appropriate transcriptional and/or translational control signals that are operably linked to the coding genes. The control signals comprise promoter sequences, untranslated regions, ribosome binding sites, and terminator sequences. The expression modules can contain elements for expression of one single recombinant gene but can also contain elements for expression of more recombinant genes or can be organized in an operon structure for integrated expression of two or more recombinant genes. The polynucleotides may be produced by recombinant DNA technology using techniques well-known in the art. Methods that are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred aspect of the disclosure, the cell is modified with one or more expression modules. The expression modules can be integrated in the genome of the cell or can be presented to the cell on a vector. The vector can be present in the form of a plasmid, cosmid, phage, liposome, or virus, which is to be stably transformed/transfected into the metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the disclosure. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. The recombinant gene is involved in the expression of a polypeptide acting in the synthesis of a 6′sialylated disaccharide and/or 6′sialylated oligosaccharide, such as, e.g., 6′sialyllactose; or the recombinant gene is linked to other pathways in the cell that are not involved in the synthesis of a 6′sialylated disaccharide and/or 6′sialylated oligosaccharide. The recombinant genes encode endogenous proteins with a modified expression or activity, preferably the endogenous proteins are overexpressed; or the recombinant genes encode heterologous proteins that are heterogeneously introduced and expressed in the modified cell, preferably overexpressed. The endogenous proteins can have a modified expression in the cell that also expresses a heterologous protein.

According to a preferred aspect of the disclosure, the expression of each of the expression modules is constitutive or tunable as defined herein.

In a further aspect of the method and/or cell of the disclosure, the cell is modified in the expression or activity of at least one of the alpha-2,6-sialyltransferase. In a preferred embodiment, the alpha-2,6-sialyltransferase is an endogenous protein of the cell with a modified expression or activity, preferably, the endogenous alpha-2,6-sialyltransferase is overexpressed; alternatively, the alpha-2,6-sialyltransferase is a heterologous protein that is heterogeneously introduced and expressed in the cell, preferably overexpressed. The endogenous alpha-2,6-sialyltransferase can have a modified expression in the cell that also expresses a heterologous alpha-2,6-sialyltransferase.

According to a preferred aspect of the method and/or cell of the disclosure, the cell comprises a pathway for production of a 6′sialylated disaccharide and/or 6′sialylated oligosaccharide, preferably 6′sialyllactose, comprising at least one alpha-2,6-sialyltransferase according to disclosure. According to another preferred aspect of the method and/or cell of the disclosure, the pathway for production of a 6′sialylated disaccharide and/or 6′sialylated oligosaccharide, preferably 6′sialyllactose further comprises at least one enzyme chosen from the list comprising Neu5Ac synthase, N-acylglucosamine 2-epimerase, UDP-N-acetylglucosamine 2-epimerase, N-acetylmannosamine-6-phosphate 2-epimerase, bifunctional UDP-GlcNAc 2-epimerase/kinase, N-acylneuraminate-9-phosphate synthetase, phosphatase and CMP-sialic acid synthase.

In a preferred embodiment, the cell comprises a pathway for production of a 6′sialylated disaccharide and/or 6′sialylated oligosaccharide, preferably 6′sialyllactose, wherein the cell expresses at least one enzyme chosen from the list comprising an N-acylglucosamine 2-epimerase like is known, e.g., from several species including Bacteroides ovatus, E. coli, Homo sapiens, Rattus norvegicus, a Neu5Ac synthase, a CMP sialic acid synthase like is known, e.g., from Neisseria meningitidis, and an alpha-2,6-sialyltransferase according to disclosure, wherein the enzymes are as defined herein. N-acyl-D-glucosamine (GlcNAc) can be added to the cell and/or can be provided by an enzyme expressed in the cell or by the mechanism of the cell. Such cell producing GlcNAc can express a phosphatase converting GlcNAc-6-phosphate into GlcNAc, like any one or more of, e.g., the E. coli HAD-like phosphatase genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU, PsMupP from Pseudomonas putida, ScDOG1 from S. cerevisiae and BsAraL from Bacillus subtilis as described in WO18122225. Preferably, the cell is modified to produce GlcNAc. More preferably, the cell is modified for enhanced GlcNAc production. The modification can be any one or more chosen from the group comprising knockout of a glucosamine-6-phosphate deaminase, an N-acetylglucosamine-6-phosphate deacetylase and/or an N-acetyl-D-glucosamine kinase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a 6′sialylated disaccharide and/or 6′sialylated oligosaccharide, preferably 6′sialyllactose wherein the cell expresses at least one enzyme chosen from the list comprising a UDP-N-acetylglucosamine 2-epimerase like is known, e.g., from several species including Campylobacter jejuni, E. coli, Neisseria meningitidis, Bacillus subtilis, Citrobacter rodentium, a Neu5Ac synthase, a CMP sialic acid synthase like is known, e.g., from Neisseria meningitidis, and an alpha-2,6-sialyltransferase according to disclosure, wherein the enzymes are as defined herein. UDP-N-acetylglucosamine (UDP-GlcNAc) can be added to the cell and/or can be provided by an enzyme expressed in the cell or by the metabolism of the cell. Such cell producing a UDP-GlcNAc can express enzymes converting, e.g., GlcNAc, which is to be added to the cell, to UDP-GlcNAc. These enzymes may be any one or more enzymes chosen from the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. The modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

In an alternative and/or additional preferred embodiment, the cell comprises a pathway for production of a 6′sialylated disaccharide and/or 6′sialylated oligosaccharide, preferably 6′sialyllactose, wherein the cell expresses at least one enzyme chosen from the list comprising an N-acetylmannosamine-6-phosphate 2-epimerase like is known, e.g., from several species including E. coli, Haemophilus influenzae, Enterobacter sp., Streptomyces sp., an N-acylneuraminate-9-phosphate synthetase, an N-acylneuraminate-9-phosphate phosphatase like is known, e.g., from Candidatus Magnetomorum sp. HK-1 or Bacteroides thetaiotaomicron, a Neu5Ac synthase, a CMP sialic acid synthase like is known, e.g., from Neisseria meningitidis, and an alpha-2,6-sialyltransferase according to disclosure, wherein the enzymes are as defined herein. N-acetyl-D-glucosamine 6-phosphate (GlcNAc-6P) can be added to the cell and/or can be provided by an enzyme expressed in the cell or by the metabolism of the cell. Such cell producing GlcNAc-6P can express an enzyme converting, e.g., GlcN6P, which is to be added to the cell, to GlcNAc-6P. This enzyme may be a glucosamine 6-phosphate N-acetyltransferase from several species including Saccharomyces cerevisiae, Kluyveromyces lactis, Homo sapiens. Preferably, the cell is modified to produce GlcNAc-6P. More preferably, the cell is modified for enhanced GlcNAc-6P production. The modification can be any one or more chosen from the group comprising knockout of a glucosamine-6-phosphate deaminase, an N-acetylglucosamine-6-phosphate deacetylase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a 6′sialylated disaccharide and/or 6′sialylated oligosaccharide, preferably 6′sialyllactose wherein the cell expresses at least one enzyme chosen from the list comprising a bifunctional UDP-GlcNAc 2-epimerase/kinase like is known, e.g., from several species including Homo sapiens, Rattus norvegicus and Mus musculus, an N-acylneuraminate-9-phosphate synthetase, an N-acylneuraminate-9-phosphate phosphatase like is known, e.g., from Candidatus Magnetomorum sp. HK-1 or Bacteroides thetaiotaomicron, a Neu5Ac synthase, a CMP sialic acid synthase like is known, e.g., from Neisseria meningitidis, and an alpha-2,6-sialyltransferase according to disclosure, wherein the enzymes are as defined herein. UDP-N-acetylglucosamine can be added to the cell and/or can be provided by an enzyme expressed in the cell or by the metabolism of the cell. Such cell producing a UDP-GlcNAc can express enzymes converting, e.g., GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. The modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the cell used herein is optionally genetically modified to import an acceptor in the cell, by the introduction and/or overexpression of a transporter able to import the respective acceptor in the cell. Such transporter is, for example, a membrane protein belonging to the major facilitator superfamily (MFS), the ATP-binding cassette (ABC) transporter family or the PTS system involved in the uptake of, e.g., mono-, di- and/or oligosaccharides.

Additionally, or alternatively, the cell used herein is optionally genetically modified to produce polyisoprenoid alcohols like, e.g., phosphorylated dolichol that can act as lipid carrier.

Additionally, or alternatively, the cell used herein is optionally genetically modified to import lactose in the cell, by the introduction and/or overexpression of a lactose permease. The lactose permease is, for example, encoded by the lacY gene or the lac12 gene.

Additionally, or alternatively, the cell expresses a membrane protein that is a transporter protein involved in transport of compounds and/or a 6′sialylated disaccharide and/or 6′sialylated oligosaccharide as defined in disclosure across the outer membrane of the cell wall. Preferably the cell is transformed to comprise at least one nucleic acid sequence encoding a protein selected from the group comprising a lactose transporter like, e.g., the LacY or lac12 permease, a glucose transporter, a galactose transporter, a transporter for a nucleotide-activated sugar like, for example, a transporter for UDP-GlcNAc, a transporter protein involved in transport of a 6′sialylated disaccharide and/or 6′sialylated oligosaccharide, preferably 6′sialyllactose, across the outer membrane of the cell wall. Preferably the cell is transformed to comprise at least one nucleic acid sequence encoding a membrane transporter protein selected from the group comprising a siderophore exporter, a major facilitator superfamily (MFS) transporter, an ATP-binding cassette (ABC) transporter or a sugar efflux transporter.

According to another and/or additional preferred embodiment of the method and/or cell of the disclosure, the cell produces a 6′sialylated disaccharide and/or 6′sialylated oligosaccharide intracellularly and a fraction or substantially all of the produced 6′sialylated disaccharide and/or 6′sialylated oligosaccharide remains intracellularly and/or is excreted outside the cell via passive or active transport.

According to another and/or additional preferred embodiment, the cell is further genetically modified for:
i) modified expression of an endogenous membrane protein, and/or
ii) modified activity of an endogenous membrane protein, and/or
iii) expression of a homologous membrane protein, and/or
iv) expression of a heterologous membrane protein,
wherein the membrane protein is involved in the secretion of a 6′sialylated disaccharide and/or 6′sialylated oligosaccharide outside the cell.

According to another and/or additional preferred embodiment, the cell is further genetically modified for:
i) modified expression of an endogenous membrane protein, and/or
ii) modified activity of an endogenous membrane protein, and/or
iii) expression of a homologous membrane protein, and/or
iv) expression of a heterologous membrane protein,
wherein the membrane protein is involved in the uptake of a precursor and/or an acceptor for the synthesis of a 6′sialylated disaccharide and/or 6′sialylated oligosaccharide. In a more preferred embodiment, the membrane protein is involved in the uptake of all of the required precursors. In another more preferred embodiment, the membrane protein is involved in the uptake of all of the acceptors.

According to a further preferred embodiment, the membrane protein is chosen from the list comprising porters, P—P-bond-hydrolysis-driven transporters, β-barrel porins, auxiliary transport proteins, putative transport proteins and phosphotransfer-driven group translocators. In a more preferred embodiment, the porters comprise MFS transporters, sugar efflux transporters and siderophore exporters. In another more preferred embodiment, the P—P-bond-hydrolysis-driven transporters comprise ABC transporters and siderophore exporters.

According to another preferred aspect of the method and/or cell of the disclosure, the cell is capable of synthesizing and/or synthesizes N-acetylmannosamine (ManNAc), N-acetylmannosamine-6-phosphate (ManNAc-6-phosphate) and/or phosphoenolpyruvate (PEP).

In a preferred embodiment, the cell comprises a pathway for production of a 6′sialylated disaccharide and/or 6′sialylated oligosaccharide, preferably 6′sialyllactose, comprising a pathway for production of ManNAc. ManNAc can be provided by an enzyme expressed in the cell or by the mechanism of the cell. Such cell producing ManNAc can express an N-acylglucosamine 2-epimerase like is known, e.g., from several species including Bacteroides ovatus, E. coli, Homo sapiens, Rattus norvegicus that converts GlcNAc into ManNAc. Alternatively, and/or additionally, the cell producing ManNAc can express a UDP-N-acetylglucosamine 2-epimerase like is known, e.g., from several species including Campylobacter jejuni, E. coli, Neisseria meningitidis, Bacillus subtilis, Citrobacter rodentium that converts UDP-GlcNAc into ManNAc. GlcNAc and/or UDP-GlcNAc can be added to the cell and/or provided by an enzyme expressed in the cell or by the mechanism of the cell as described herein.

In a more preferred embodiment, the cell is modified for enhanced ManNAc production. The modification can be any one or more chosen from the group comprising knock-out of N-acetylmannosamine kinase, over-expression of N-acetylneuraminate lyase.

In another preferred embodiment, the cell comprises a pathway for production of a 6′sialylated disaccharide and/or 6′sialylated oligosaccharide, preferably 6′sialyllactose, comprising a pathway for production of ManNAc-6-phosphate. ManNAc-6-phosphate can be provided by an enzyme expressed in the cell or by the mechanism of the cell. Such cell producing ManNAc-6-phosphate can express a bifunctional UDP-GlcNAc 2-epimerase/kinase like is known, e.g., from several species including Homo sapiens, Rattus norvegicus and Mus musculus that converts UDP-GlcNAc into ManNAc-6-phosphate. Alternatively, and/or additionally, the cell producing ManNAc-6-phosphate can express an N-acetylmannosamine-6-phosphate 2-epimerase that converts GlcNAc-6-phosphate into ManNAc-6-phosphate. UDP-GlcNAc and/or GlcNAc-6-phosphate can be added to the cell and/or provided by an enzyme expressed in the cell or by the mechanism of the cell as described herein. In a more preferred embodiment, the cell is modified for enhanced ManNAc-6-phosphate production. The modification can be any one or more chosen from the group comprising over-expression of N-acetylglucosamine-6-phosphate deacetylase, over-expression of N-acetyl-D-glucosamine kinase, over-expression of phosphoglucosamine mutase, over-expression of N-acetylglucosamine-1-phosphate uridyltransferase/glucosamine-1-phosphate acetyltransferase.

In another preferred embodiment, the cell comprises a pathway for production of a 6′sialylated disaccharide and/or 6′sialylated oligosaccharide comprising a pathway for production of phosphoenolpyruvate (PEP).

In another preferred embodiment, the cell comprises a pathway for production of a 6′sialylated disaccharide and/or 6′sialylated oligosaccharide comprising any one or more modifications for enhanced production and/or supply of PEP compared to a non-modified progenitor.

In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is, for instance, encoded by the nagE gene (or the cluster nagABCD) in E. coli or Bacillus species, 2) ManXYZ, which encodes the Enzyme ll Man complex (mannose PTS permease, protein-Npi-phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance, encoded by PtsG/Crr), which takes up glucose and forms glucose-6-phosphate in the cytoplasm, 4) the sucrose-specific PTS transporter, which takes up sucrose and forms sucrose-6-phosphate in the cytoplasm, 5) the fructose-specific PTS transporter (for instance, encoded by the genes fruA and fruB and the kinase fruK, which takes up fructose and forms in a first step fructose-1-phosphate and in a second step fructose1,6 bisphosphate, 6) the lactose PTS transporter (for instance, encoded by lack in Lactococcus casei) which, takes up lactose and forms lactose-6-phosphate, 7) the galactitol-specific PTS enzyme, which takes up galactitol and/or sorbitol and forms galactitol-1-phosphate or sorbitol-6-phosphate respectively, 8) the mannitol-specific PTS enzyme, which takes up mannitol and/or sorbitol and forms mannitol-1-phosphate or sorbitol-6-phosphate respectively, and 9) the trehalose-specific PTS enzyme, which takes up trehalose and forms trehalose-6-phosphate.

In another and/or additional preferred embodiment and as a means for enhanced production and/or supply of PEP, the full PTS system is disrupted by disrupting the PtsIH/Crr gene cluster. PtsI (Enzyme I) is a cytoplasmic protein that serves as the gateway for the phosphoenolpyruvate: sugar phosphotransferase system (PTS^{sugar}) of E. coli K-12. PtsI is one of two (PtsI and PtsH) sugar non-specific protein constituents of the PTS^{sugar}, which along with a sugar-specific inner membrane permease, effects a phosphotransfer cascade that results in the coupled phosphorylation and transport of a variety of carbohydrate substrates. HPr (histidine containing protein) is one of two sugar-non-specific protein constituents of the PTS^{sugar}. It accepts a phosphoryl group from phosphorylated Enzyme I (PtsI-P) and then transfers it to the EIIA domain of any one of the many sugar-specific enzymes (collectively known as Enzymes II) of the PTS^{sugar} Crr or EIIA^{Gle} is phosphorylated by PEP in a reaction requiring PtsH and PtsI.

In another and/or additional preferred embodiment, the cell is further modified to compensate for the deletion of a PTS system of a carbon source by the introduction and/or overexpression of the corresponding permease. These are, e.g., permeases or ABC transporters that comprise but are not limited to transporters that specifically import lactose such as, e.g., the transporter encoded by the LacY gene from E. coli, sucrose such as, e.g., the transporter encoded by the cscB gene from E. coli, glucose such as, e.g., the transporter encoded by the gall gene from E. coli, fructose such as, e.g., the transporter encoded by the fruI gene from Streptococcus mutans, or the Sorbitol/mannitol ABC transporter such as the transporter encoded by the cluster SmoEFGK of Rhodobacter sphaeroides, the trehalose/sucrose/maltose transporter such as the transporter encoded by the gene cluster ThuEFGK of Sinorhizobium meliloti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g., ptsG gene, combined with the introduction and/or overexpression of a glucose permease (e.g., galP of glcP), 2) the deletion of the fructose PTS system, e.g., one or more of the fruB, fruA, fruK genes, combined with the introduction and/or overexpression of fructose permease, e.g., fruI, 3) the deletion of the lactose PTS system, combined with the introduction and/or overexpression of lactose permease, e.g., LacY, and/or 4) the deletion of the sucrose PTS system, combined with the introduction and/or overexpression of a sucrose permease, e.g., cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4). Examples of combinations of PTS deletions with overexpression of alternative transporters and a kinase are: 1) the deletion of the glucose PTS system, e.g., ptsG gene, combined with the introduction and/or overexpression of a glucose permease (e.g., galP of glcP), combined with the introduction and/or overexpression of a glucokinase (e.g., glk), and/or 2) the deletion of the fructose PTS system, e.g., one or more of the fruB, fruA, fruK genes, combined with the introduction and/or overexpression of fructose permease, e.g., fruI, combined with the introduction and/or overexpression of a fructokinase (e.g., frk or mak).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded for instance in E. coli by ppsA), phosphoenolpyruvate carboxykinase activity (EC 4.1.1.32 or EC 4.1.1.49 encoded for instance in Corynebacterium glutamicum by PCK or in E. coli by pckA, resp.), phosphoenolpyruvate carboxylase activity (EC 4.1.1.31 encoded for instance in E. coli by ppc), oxaloacetate decarboxylase activity (EC 4.1.1.112 encoded for instance in E. coli by eda), pyruvate kinase activity (EC 2.7.1.40 encoded for instance in E. coli by pykA and pykF), pyruvate carboxylase activity (EC 6.4.1.1 encoded for instance in B. subtilis by pyc) and malate dehydrogenase activity (EC 1.1.1.38 or EC 1.1.1.40 encoded for instance in E. coli by maeA or maeB, resp.).

In a more preferred embodiment, the cell is modified to overexpress any one or more of the polypeptides comprising ppsA from E. coli with SEQ ID NO: 41, PCK from C. glutamicum with SEQ ID NO: 42, pcka from E. coli with SEQ ID NO: 43, eda from E. coli with SEQ ID NO: 44, maeA from E. coli with SEQ ID NO: 45 and maeB from E. coli with SEQ ID NO: 46.

In another and/or additional preferred embodiment, the cell is modified to express any one or more of a functional homolog, variant or derivative of any one of SEQ ID NOS: 41, 42, 43, 44, 45 or 46 having at least 80% overall sequence identity to the full-length of any one of the polypeptide with SEQ ID NOS: 41, 42, 43, 44, 45 or 46, and having phosphoenolpyruvate synthase activity, phosphoenolpyruvate carboxykinase activity, oxaloacetate decarboxylase activity, or malate dehydrogenase activity, respectively.

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of the genes encoding for phosphoenolpyruvate carboxylase, the pyruvate carboxylase activity and/or pyruvate kinase.

In an exemplary embodiment, the cell is genetically modified by different adaptations such as the overexpression of phosphoenolpyruvate synthase combined with the deletion of a pyruvate kinase gene, the overexpression of phosphoenolpyruvate synthase combined with the deletion of a phosphoenolpyruvate carboxylase gene, the overexpression of phosphoenolpyruvate synthase combined with the deletion of a pyruvate carboxylase gene, the overexpression of phosphoenolpyruvate carboxykinase combined with the deletion of a pyruvate kinase gene, the overexpression of phosphoenolpyruvate carboxykinase combined with the deletion of a phosphoenolpyruvate carboxylase gene, the overexpression of phosphoenolpyruvate carboxykinase combined with the deletion of a pyruvate carboxylase gene, the overexpression of oxaloacetate decarboxylase combined with the deletion of a pyruvate kinase gene, the overexpression of oxaloacetate decarboxylase combined with the deletion of a phosphoenolpyruvate carboxylase gene, the overexpression of oxaloacetate decarboxylase combined with the deletion of a pyruvate carboxylase gene, the overexpression of malate dehydrogenase combined with the deletion of a pyruvate kinase gene, the overexpression of malate dehydrogenase combined with the deletion of a phosphoenolpyruvate carboxylase gene and/or the overexpression of malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase, the overexpression of phosphoenolpyruvate synthase combined with the overexpression of an oxaloacetate decarboxylase, the overexpression of phosphoenolpyruvate synthase combined with the overexpression of a malate dehydrogenase, the overexpression of phosphoenolpyruvate carboxykinase combined with the overexpression of an oxaloacetate decarboxylase, the overexpression of phosphoenolpyruvate carboxykinase combined with the overexpression of a malate dehydrogenase, the overexpression of oxaloacetate decarboxylase combined with the overexpression of a malate dehydrogenase, the overexpression of phosphoenolpyruvate synthase combined with the overexpression of a phosphoenolpyruvate carboxykinase and the overexpression of an oxaloacetate decarboxylase, the overexpression of phosphoenolpyruvate synthase combined with the overexpression of a phosphoenolpyruvate carboxykinase and the overexpression of a malate dehydrogenase, the overexpression of phosphoenolpyruvate synthase combined with the overexpression of a phosphoenolpyruvate carboxykinase and the overexpression of an oxaloacetate decarboxylase and the overexpression of a malate dehydrogenase, the overexpression of phosphoenolpyruvate carboxykinase combined with the overexpression of an oxaloacetate decarboxylase and the overexpression of a malate dehydrogenase and/or the overexpression of phosphoenolpyruvate synthase combined with the overexpression of an oxaloacetate decarboxylase and the overexpression of a malate dehydrogenase.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene, the overexpression of phosphoenolpyruvate synthase combined with the overexpression of an oxaloacetate decarboxylase combined with the deletion of a pyruvate kinase gene, the overexpression of phosphoenolpyruvate synthase combined with the overexpression of a malate dehydrogenase combined with the deletion of a pyruvate kinase gene, the overexpression of phosphoenolpyruvate carboxykinase combined with the overexpression of an oxaloacetate decarboxylase combined with the deletion of a pyruvate kinase gene, the overexpression of phosphoenolpyruvate carboxykinase combined with the overexpression of a malate dehydrogenase combined with the deletion of a pyruvate kinase gene, the overexpression of an oxaloacetate decarboxylase combined with the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene, the overexpression of phosphoenolpyruvate synthase combined with the overexpression of a phosphoenolpyruvate carboxykinase and the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene, the overexpression of a phosphoenolpyruvate carboxykinase combined with the overexpression of an oxaloacetate decarboxylase and the overexpression of a malate dehydrogenase combined with the deletion of a pyruvate kinase gene, the overexpression of phosphoenolpyruvate synthase combined the overexpression of oxaloacetate decarboxylase and the overexpression of a malate dehydrogenase combined with the deletion of a pyruvate kinase gene.

In another exemplary embodiment, the cell is genetically modified by different adaptations such as the overexpression of phosphoenolpyruvate synthase combined with the overexpression of a phosphoenolpyruvate carboxykinase combined with the deletion of a phosphoenolpyruvate carboxylase gene, the overexpression of phosphoenolpyruvate synthase combined with the overexpression of an oxaloacetate decarboxylase combined with the deletion of a phosphoenolpyruvate carboxylase gene, the overexpression of phosphoenolpyruvate synthase combined with the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase combined with the deletion of a phosphoenolpyruvate carboxylase gene, the overexpression of a phosphoenolpyruvate carboxykinase combined with the overexpression of a malate dehydrogenase combined with the deletion of a phosphoenolpyruvate carboxylase gene, the overexpression of an oxaloacetate decarboxylase combined with the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a phosphoenolpyruvate carboxylase gene, the overexpression of phosphoenolpyruvate synthase combined the overexpression of an oxaloacetate decarboxylase and the overexpression of a malate dehydrogenase combined with the deletion of a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate carboxylase gene, the overexpression of phosphoenolpyruvate synthase combined with the overexpression of an oxaloacetate decarboxylase combined with the deletion of a pyruvate carboxylase gene, the overexpression of phosphoenolpyruvate synthase combined with the overexpression of a malate dehydrogenase combined with the deletion of a pyruvate carboxylase gene, the overexpression of a phosphoenolpyruvate carboxykinase combined with the overexpression of an oxaloacetate decarboxylase combined with the deletion of a pyruvate carboxylase gene, the overexpression of a phosphoenolpyruvate carboxykinase combined with the overexpression of a malate dehydrogenase combined with the deletion of a pyruvate carboxylase gene, the overexpression of an oxaloacetate decarboxylase combined with the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate carboxylase gene, the overexpression of phosphoenolpyruvate synthase combined with the overexpression of an oxaloacetate decarboxylase and the overexpression of a malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a phosphoenolpyruvate carboxylase gene, the overexpression of phosphoenolpyruvate synthase combined with the overexpression of an oxaloacetate decarboxylase combined with the deletion of a pyruvate kinase gene and a phosphoenolpyruvate carboxylase gene, the overexpression of phosphoenolpyruvate synthase combined with the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a phosphoenolpyruvate carboxylase gene, the overexpression of a phosphoenolpyruvate carboxykinase combined with the overexpression of a malate dehydrogenase combined with the deletion of a pyruvate kinase gene and a phosphoenolpyruvate carboxylase gene, the overexpression of an oxaloacetate decarboxylase combined with the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate synthase combined with the overexpression of an oxaloacetate decarboxylase and the overexpression of a malate dehydrogenase combined with the deletion of a pyruvate kinase gene and a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a malate dehydrogenase combined with the deletion of a pyruvate kinase gene and a pyruvate carboxylase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a pyruvate carboxylase gene and a phosphoenolpyruvate carboxylase gene, the overexpression of a phosphoenolpyruvate carboxykinase combined with the overexpression of a malate dehydrogenase combined with the deletion of a pyruvate kinase gene and a pyruvate carboxylase gene and a phosphoenolpyruvate carboxylase gene, the overexpression of an oxaloacetate decarboxylase combined with the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and pyruvate carboxylase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene.

According to another preferred aspect of the method and/or cell of the disclosure, the cell comprises a modification for reduced production of acetate compared to a non-modified progenitor. The modification can be any one or more chosen from the group comprising overexpression of an acetyl-coenzyme A synthetase, a full or partial knock-out or rendered less functional pyruvate dehydrogenase and a full or partial knock-out or rendered less functional lactate dehydrogenase.

In a further aspect of the method and/or cell of the disclosure, the cell is modified in the expression or activity of at least one acetyl-coenzyme A synthetase like, e.g., acs from E. coli, S. cerevisiae, H. sapiens, M. musculus. In a preferred embodiment, the acetyl-coenzyme A synthetase is an endogenous protein of the cell with a modified expression or activity, preferably, the endogenous acetyl-coenzyme A synthetase is overexpressed; alternatively, the acetyl-coenzyme A synthetase is a heterologous protein that is heterogeneously introduced and expressed in the cell, preferably overexpressed. The endogenous acetyl-coenzyme A synthetase can have a modified expression in the cell, which also expresses a heterologous acetyl-coenzyme A synthetase.

In an alternative and/or additional further aspect of the method and/or cell of the disclosure, the cell is modified in the expression or activity of at least one pyruvate dehydrogenase like, e.g., from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further aspect of the method and/or cell of the disclosure, the cell is modified in the expression or activity of at least one lactate dehydrogenase like, e.g., from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the ldhA encoding gene resulting in a cell lacking lactate dehydrogenase activity.

According to another preferred aspect of the method and/or cell of the disclosure, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Gle}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, and pyruvate decarboxylase.

According to another aspect of the method and/or cell of the disclosure, the cell is further capable to synthesize any one or more nucleotide-activated sugars. In a preferred embodiment of the method and/or cell of the disclosure, the cell is capable to synthesize one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), GDP-fucose (GDP-Fuc), GDP-rhamnose and UDP-xylose. In a more preferred embodiment of the method and/or cell of the disclosure, the cell is capable of synthesizing at least nucleotide-activated sugar CMP-Neu5Ac. In an even more preferred embodiment of the method and/or cell of the disclosure, the cell uses at least one of the synthesized nucleotide-activated sugars in the production of a 6′sialylated disaccharide and/or 6′sialylated oligosaccharide, preferably 6′sialyllactose.

The cell used herein is optionally genetically modified to express the de novo synthesis of UDP-GlcNAc. UDP-GlcNAc can be provided by an enzyme expressed in the cell or by the metabolism of the cell. Such cell producing a UDP-GlcNAc can express enzymes converting, e.g., GlcNAc, which is to be added to the cell, to UDP-GlcNAc. These enzymes may be any one or more of the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. The modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the cell used herein is optionally genetically modified to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5 Ac production. The modification can be any one or more chosen from the group comprising knock-out of an N-acetylglucosamine-6-phosphate deacetylase, knock-out of a glucosamine-6-phosphate deaminase, over-expression of a CMP-sialic acid synthetase, and over-expression of an N-acetyl-D-glucosamine-2-epimerase encoding gene.

Additionally, or alternatively, the cell used herein is optionally genetically modified to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. The modification can be any one or more chosen from the group comprising knock-out of a UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the cell used herein is optionally genetically modified to express the de novo synthesis of UDP-Gal. UDP-Gal can be provided by an enzyme expressed in the cell or by the metabolism of the cell. Such cell producing UDP-Gal can express an enzyme converting, e.g., UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. The modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of a UDP-glucose-4-epimerase encoding gene.

Additionally, or alternatively, the cell used herein is optionally genetically modified to express the de novo synthesis of UDP-GalNAc. UDP-GalNAc can be synthesized from UDP-GlcNAc by the action of a single-step reaction using a UDP-N-acetylglucosamine 4-epimerase like, e.g., wbgU from Plesiomonas shigelloides, gne from Yersinia enterocolitica or wbpP from Pseudomonas aeruginosa serotype O6. Preferably, the cell is modified to produce UDP-GalNAc. More preferably, the cell is modified for enhanced UDP-GalNAc production.

Additionally, or alternatively, the cell used herein is optionally genetically modified to express the de novo synthesis of UDP-ManNAc. UDP-ManNAc can be synthesized directly from UDP-GlcNAc via an epimerization reaction performed by a UDP-GlcNAc 2-epimerase (like, e.g., cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

According to another aspect of the method and/or cell of the disclosure, the cell expresses at least one further glycosyltransferase chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a preferred embodiment of the method and/or cell of the disclosure, the fucosyltransferase is chosen from the list comprising alpha-1,2-fucosyltransferase, alpha-1,3-fucosyltransferase, alpha-1,4-fucosyltransferase and alpha-1,6-fucosyltransferase.

In an alternative and/or additional embodiment of the method and/or cell of the disclosure, the further sialyltransferase is chosen from the list comprising alpha-2,3-sialyltransferase, alpha-2,6-sialyltransferase, and alpha-2,8-sialyltransferase.

In an alternative and/or additional embodiment of the method and/or cell of the disclosure, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase, and alpha-1,4-galactosyltransferase.

In an alternative and/or additional embodiment of the method and/or cell of the disclosure, the glucosyltransferase is chosen from the list comprising alpha-glucosyltransferase, beta-1,2-glucosyltransferase, beta-1,3-glucosyltransferase and beta-1,4-glucosyltransferase.

In an alternative and/or additional embodiment of the method and/or cell of the disclosure, the mannosyltransferase is chosen from the list comprising alpha-1,2-mannosyltransferase, alpha-1,3-mannosyltransferase and alpha-1,6-mannosyltransferase.

In an alternative and/or additional embodiment of the method and/or cell of the disclosure, the N-acetylglucosaminyltransferase is chosen from the list comprising galactoside beta-1,3-N-acetylglucosaminyltransferase and beta-1,6-N-acetylglucosaminyltransferase.

In an alternative and/or additional embodiment of the method and/or cell of the disclosure, the N-acetylgalactosaminyltransferase is chosen from the list comprising alpha-1,3-N-acetylgalactosaminyltransferase.

In a further aspect of the method and/or cell of the disclosure, the cell is modified in the expression or activity of at least one of the glycosyltransferases. In a preferred embodiment, the glycosyltransferase is an endogenous protein of the cell with a modified expression or activity, preferably, the endogenous glycosyltransferase is overexpressed; alternatively, the glycosyltransferase is a heterologous protein that is heterogeneously introduced and expressed in the cell, preferably overexpressed. The endogenous glycosyltransferase can have a modified expression in the cell, which also expresses a heterologous glycosyltransferase.

According to another and/or alternative preferred aspect of the method and/or cell of the disclosure, the cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, fucosyltransferase.

According to another and/or alternative preferred aspect of the method and/or cell of the disclosure, the cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase, galactosyltransferase.

According to another and/or alternative preferred aspect of the method and/or cell of the disclosure, the cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.

In an alternative and/or additional further aspect of the method and/or cell of the disclosure, the cell is modified in the expression or activity of at least one pyruvate dehydrogenase like, e.g., from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further aspect of the method and/or cell of the disclosure, the cell is modified in the expression or activity of at least one lactate dehydrogenase like, e.g., from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the ldhA encoding gene resulting in a cell lacking lactate dehydrogenase activity.

According to another preferred aspect of the method and/or cell of the disclosure, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose: undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Gle}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred aspect of the method and/or cell of the disclosure, the cell is using a precursor for the synthesis of 6′sialylated disaccharide and/or 6′sialylated oligosaccharide. Herein, the precursor is fed to the cell from the cultivation medium. In another preferred embodiment, the cell is producing a precursor for the synthesis of the 6′sialylated disaccharide and/or 6′sialylated oligosaccharide.

According to another preferred aspect of the method and/or cell of the disclosure, the cell produces 90 g/L or more of a 6′sialylated disaccharide and/or 6′sialylated oligosaccharide in the whole broth and/or supernatant. In a more preferred embodiment, a 6′sialylated disaccharide and/or 6′sialylated oligosaccharide produced in the whole broth and/or supernatant has a purity of at least 80% measured on the total amount of the 6′sialylated disaccharide and/or 6′sialylated oligosaccharide and its precursor produced by the cell in the whole broth and/or supernatant, respectively.

According to another aspect of the method and/or cell of the disclosure, the 6′sialylated oligosaccharide is chosen from the list comprising a milk oligosaccharide, O-antigen, enterobacterial common antigen (ECA), the oligosaccharide repeats present in capsular polysaccharides, amino-sugars, and antigens of the human ABO blood group system. In a more preferred embodiment, the milk oligosaccharide is a mammalian milk oligosaccharide. In an even more preferred embodiment, the milk oligosaccharide is a human milk oligosaccharide.

According to another aspect of the method and/or cell of the disclosure, the cell is capable to synthesize a mixture of oligosaccharides. In an alternative and/or additional aspect, the cell is capable of synthesizing a mixture of di- and oligosaccharides; alternatively, the cell is capable of synthesizing a mixture of sialic acid, di- and/or oligosaccharides.

Another aspect of the disclosure provides for a method and a cell wherein a 6′sialylated disaccharide and/or 6′sialylated oligosaccharide is produced in and/or by a cell, which is a bacterium, fungus, yeast, a plant cell, an animal cell, or a protozoan cell. The latter bacterium preferably belongs to the phylum of the Proteobacteria or the phylum of the Firmicutes or the phylum of the Cyanobacteria or the phylum Deinococcus-Thermus or the phylum of Actinobacteria. The latter bacterium belonging to the phylum Proteobacteria belongs preferably to the family Enterobacteriaceae, preferably to the species Escherichia coli. Preferably, the bacterium is an Escherichia coli strain such as but not limited to Escherichia coli B, Escherichia coli C, Escherichia coli W, Escherichia coli K12, Escherichia coli Nissle, more preferably an Escherichia coli strain, which is a K-12 strain. Well-known examples of the E. coli K12 strains are K12 Wild type, W3110, MG1655, M182, MC1000, MC1060, MC1061, MC4100, JM101, NZN111 and AA200. Even more preferably the Escherichia coli K-12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter bacterium belonging to the phylum Proteobacteria, preferably belonging to the family of the Vibrionaceae, with member Vibrio natriegens. Preferably, the fungus belongs to a genus chosen from the group comprising Rhizopus, Dictyostelium, Penicillium, Mucor or Aspergillus. The latter yeast preferably belongs to the phylum of the Ascomycota or the phylum of the Basidiomycota or the phylum of the Deuteromycota or the phylum of the Zygomycetes. Preferably, the yeast belongs to a genus chosen from the group comprising Saccharomyces (with members like, e.g., Saccharomyces cerevisiae, S. bayanus, S. boulardii), Zygosaccharomyces, Pichia (with members like, e.g., Pichia pastoris, P. anomala, P. kluyveri), Komagataella, Hansenula, Candida, Schizosaccharomyces, Schwanniomyces, Torulaspora, Yarrowia like, e.g., Yarrowia lipolytica), Starmerella like, e.g., Starmerella bombicola), Kluyveromyces (with members like, e.g., Kluyveromyces lactis, K. marxianus, K. thermotolerans) or Debaromyces. The latter yeast is preferably selected from Pichia pastoris, Yarrowia lipolitica, Saccharomyces cerevisiae, Kluyveromyces lactis, Hansenula polymorpha, Kluyveromyces marxianus, Pichia methanolica, Pichia stipites, Candida boidinii, Schizosaccharomyces pombe, Schwanniomyces occidentalis, Torulaspora delbrueckii, Zygosaccharomyces rouxii, and Zygosaccharomyces bailii. Plant cells include cells of flowering and non-flowering plants, as well as algal cells, for example, Chlamydomonas, Chlorella, etc. Preferably, the plant cell is an algal cell or is derived from tobacco, alfalfa, rice, tomato, cotton, rapeseed, soy, maize, or corn plant. Preferably, the animal cell is derived from non-human mammals (e.g., cattle, buffalo, pig, sheep, mouse, rat, primate (e.g., chimpanzee, orangutan, gorilla, monkey (e.g., Old World, New World), lemur), dog, cat, rabbit, horse, cow, goat, ox, deer, musk deer, bovid, whale, dolphin, hippopotamus, elephant, rhinoceros, giraffe, zebra, lion, cheetah, tiger, panda, red panda, otter), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified or engineered cell line derived from human cells excluding embryonic stem cells. Both human and non-human mammalian cells are preferably chosen from the list comprising an epithelial cell like, e.g., a mammary epithelial cell, an embryonic kidney cell (e.g., HEK293 or HEK 293T cell), a fibroblast cell, a COS cell, a Chinese hamster ovary (CHO) cell, a murine myeloma cell like, e.g., an N20, SP2/0 or YB2/0 cell, an NIH-3T3 cell, a non-mammary adult stem cell or derivatives thereof such as described in WO21067641, a lactocyte derived from mammalian-induced pluripotent stem cells, preferably, human-induced pluripotent stem cells, a lactocyte as part of mammary-like gland organoids, a post-parturition mammary epithelium cell, a polarized mammary cell, preferably a polarized mammary cell selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof as well-known to the person skilled in the art from, e.g., WO 2021/219634, WO 2022/054053, WO 2021/141762, WO 2021/142241, WO 2021/067641 and WO 2021/242866. More preferably, the insect cell is derived from Spodoptera frugiperda like, e.g., Sf9 or Sf21 cells, Bombyx mori, Mamestra brassicae, Trichoplusiani like, e.g., BTI-TN-5B1-4 cells or Drosophila melanogaster like, e.g., Drosophila S2 cells. Preferably, the protozoan cell is a Leishmania tarentolae cell.

More preferably, the cell is selected from the group consisting of prokaryotic cells and eukaryotic cells, preferably from the group consisting of yeast cells, bacterial cells, archaebacterial cells, algae cells, and fungal cells as described herein.

According to another aspect of the method and/or cell of the disclosure, the cell as described herein comprises i) a sequence comprising a polynucleotide encoding the alpha-2,6-sialyltransferase, wherein the sequence is a sequence foreign to the cell and wherein the sequence is integrated in the genome of the cell, or ii) containing a vector comprising a polynucleotide encoding the alpha-2,6-sialyltransferase as described herein, wherein the polynucleotide being operably linked to control sequences recognized by a cell transformed with the vector.

According to another preferred aspect of the method and/or cell of the disclosure, the cell comprises a catabolic pathway for selected mono-, di- or oligosaccharides, which is at least partially inactivated, the mono-, di-, or oligosaccharides being involved in and/or required for the synthesis of a 6′sialylated disaccharide and/or 6′sialylated oligosaccharide. Preferably, the 6′sialylated disaccharide and/or 6′sialylated oligosaccharide is 6′sialyllactose.

A further aspect of the disclosure provides for an isolated nucleic acid molecule encoding an alpha-2,6-sialyltransferase as described herein.

Another aspect of the disclosure provides for a vector comprising an isolated nucleic acid molecule encoding an alpha-2,6-sialyltransferase as described herein.

Another aspect of the disclosure provides the use of an alpha-2,6-sialyltransferase as described herein for production of a 6′sialylated disaccharide and/or 6′sialylated oligosaccharide, preferably 6′sialyllactose. A further aspect of the disclosure provides the use of a cell as described herein for production of a 6′sialylated disaccharide and/or 6′sialylated oligosaccharide, preferably 6′sialyllactose.

Still a further aspect of the disclosure provides i) use of a method as described herein for production of a 6′sialylated disaccharide and/or 6′sialylated oligosaccharide, preferably 6′sialyllactose, ii) use of an isolated nucleic acid molecule as described herein for production of a 6′sialylated disaccharide and/or 6′sialylated oligosaccharide, preferably 6′sialyllactose, or iii) use of a vector as described herein for production of a 6′sialylated disaccharide and/or 6′sialylated oligosaccharide, preferably 6′sialyllactose.

Another aspect provides for a cell to be stably cultured in a medium, wherein the medium can be any type of growth medium comprising minimal medium, complex medium or growth medium enriched in certain compounds like, for example, but not limited to vitamins, trace elements, amino acids.

The microorganism or cell as used herein is capable to grow on a monosaccharide, disaccharide, oligosaccharide, polysaccharide, polyol, glycerol, a complex medium or a mixture thereof as the main carbon source. With the term “main” is meant the most important carbon source for the microorganism or cell for the production of the 6′sialylated disaccharide and/or 6′sialylated oligosaccharide of interest, biomass formation, carbon dioxide and/or by-products formation (such as acids and/or alcohols, such as acetate, lactate, and/or ethanol), i.e., 20, 30, 40, 50, 60, 70, 75, 80, 85, 90, 95, 98, 99% of all the required carbon is derived from the above-indicated carbon source. In one embodiment of the disclosure, the carbon source is the sole carbon source for the organism, i.e., 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of the 6′sialylated disaccharide and/or 6′sialylated oligosaccharide of the disclosure.

According to the disclosure, the methods as described herein preferably comprises a step of separating the 6′sialylated disaccharide and/or 6′sialylated oligosaccharide of the disclosure from the cultivation or incubation, otherwise the recovering the 6′sialylated disaccharide and/or 6′sialylated oligosaccharide from the cultivation or incubating medium and/or the cell.

The terms “separating from the cultivation or incubation” means harvesting, collecting, or retrieving the 6′sialylated disaccharide and/or 6′sialylated oligosaccharide from the cell and/or the medium of its growth.

The 6′sialylated disaccharide and/or 6′sialylated oligosaccharide can be separated in a conventional manner from the aqueous culture medium, in which the cell was grown. In case the 6′sialylated disaccharide and/or 6′sialylated oligosaccharide is still present in the cells producing the 6′sialylated disaccharide and/or 6′sialylated oligosaccharide, conventional manners to free or to extract the 6′sialylated disaccharide and/or 6′sialylated oligosaccharide out of the cells can be used, such as cell destruction using high pH, heat shock, sonication, French press, homogenization, enzymatic hydrolysis, chemical hydrolysis, solvent hydrolysis, detergent, hydrolysis, etc. The culture medium and/or cell extract together and separately can then be further used for separating the 6′sialylated disaccharide and/or 6′sialylated oligosaccharide.

This preferably involves clarifying the 6′sialylated disaccharide and/or 6′sialylated oligosaccharide to remove suspended particulates and contaminants, particularly cells, cell components, insoluble metabolites and debris produced by culturing the genetically modified cell. In this step, the 6′sialylated disaccharide and/or 6′sialylated oligosaccharide can be clarified in a conventional manner. Preferably, the 6′sialylated disaccharide and/or 6′sialylated oligosaccharide is clarified by centrifugation, flocculation, decantation and/or filtration. A second step of separating the 6′sialylated disaccharide and/or 6′sialylated oligosaccharide preferably involves removing substantially all the eventually remaining proteins, peptides, amino acids, RNA, DNA, endotoxins and glycolipids that could interfere with the subsequent separation step, from the 6′sialylated disaccharide and/or 6′sialylated oligosaccharide, preferably after it has been clarified. In this step, remaining proteins and related impurities can be removed from the 6′sialylated disaccharide and/or 6′sialylated oligosaccharide in a conventional manner. Preferably, remaining proteins, salts, by-products, color, endotoxins and other related impurities are removed from the 6′sialylated disaccharide and/or 6′sialylated oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including, e.g., DEAE-sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, remaining proteins and related impurities are retained by a chromatography medium or a selected membrane.

In a further preferred embodiment, the methods as described herein also provide for a further purification of the 6′sialylated disaccharide and/or 6′sialylated oligosaccharide of the disclosure. A further purification of the 6′sialylated disaccharide and/or 6′sialylated oligosaccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the 6′sialylated disaccharide and/or 6′sialylated oligosaccharide. Another purification step is to dry, e.g., spray dry or lyophilize the produced 6′sialylated disaccharide and/or 6′sialylated oligosaccharide.

In an exemplary embodiment, the separation and purification of the 6′sialylated disaccharide and/or 6′sialylated oligosaccharide is made in a process, comprising the following steps in any order:
a) contacting the cultivation or a clarified version thereof with a nanofiltration membrane with a molecular weight cut-off (MWCO) of 600-3500 Da ensuring the retention of the produced 6′sialylated disaccharide and/or 6′sialylated oligosaccharide and allowing at least a part of the proteins, salts, by-products, color and other related impurities to pass,
b) conducting a diafiltration process on the retentate from step a), using the membrane, with an aqueous solution of an inorganic electrolyte, followed by optional diafiltration with pure water to remove excess of the electrolyte,
c) and collecting the retentate enriched in the 6′sialylated disaccharide and/or 6′sialylated oligosaccharide in the form of a salt from the cation of the electrolyte.

In an alternative exemplary embodiment, the separation and purification of the 6′sialylated disaccharide and/or 6′sialylated oligosaccharide is made in a process, comprising the following steps in any order: subjecting the cultivation or a clarified version thereof to two membrane filtration steps using different membranes, wherein:
one membrane has a molecular weight cut-off of between about 300 to about 500 Dalton, and
the other membrane as a molecular weight cut-off of between about 600 to about 800 Dalton.

In an alternative exemplary embodiment, the separation and purification of the 6′sialylated disaccharide and/or 6′sialylated oligosaccharide is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of the 6′sialylated disaccharide and/or 6′sialylated oligosaccharide is made in the following way. The cultivation comprising the produced 6′sialylated disaccharide and/or 6′sialylated oligosaccharide, biomass, medium components and contaminants, and wherein the purity of the produced 6′sialylated disaccharide and/or 6′sialylated oligosaccharide in the cultivation is <80%, is applied to the following purification steps:
i) separation of biomass from the cultivation,
ii) cationic ion exchanger treatment for the removal of positively charged material,
iii) anionic ion exchanger treatment for the removal of negatively charged material, and
iv) nanofiltration step and/or electrodialysis step,
wherein a purified solution comprising the produced 6′sialylated disaccharide and/or 6′sialylated oligosaccharide at a purity of greater than or equal to 80% is provided. Optionally the purified solution is spray dried.

In an alternative exemplary embodiment, the separation and purification of the 6′sialylated disaccharide and/or 6′sialylated oligosaccharide is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises:
i) at least 4 columns, wherein at least one column comprises a weak or strong cation exchange resin; and/or
ii) four zones I, II, III and IV with different flow rates; and/or
iii) an eluent comprising water; and/or
iv) an operating temperature of 15 degrees to 60 degrees centigrade.

In a specific embodiment, the disclosure provides the produced 6′sialylated disaccharide and/or 6′sialylated oligosaccharide, which is spray-dried to powder, wherein the spray-dried powder contains <15%-wt. of water, preferably <10%-wt. of water, more preferably <7%-wt. of water, most preferably <5%-wt. of water.

Another aspect of the disclosure provides the use of a cell as defined herein, in a method for the production of a 6′sialylated disaccharide and/or 6′sialylated oligosaccharide. A further aspect of the disclosure provides the use of a method as defined herein for the production of a 6′sialylated disaccharide and/or 6′sialylated oligosaccharide.

Furthermore, the disclosure also relates to the 6′sialylated disaccharide and/or 6′sialylated oligosaccharide obtained by the methods according to the disclosure, as well as to the use of a polynucleotide, the vector, host cells, cells, microorganisms or the polypeptide as described above for the production of the 6′sialylated disaccharide and/or 6′sialylated oligosaccharide. The 6′sialylated disaccharide and/or 6′sialylated oligosaccharide may be used as food additive, prebiotic, symbiotic, for the supplementation of baby food, adult food or feed, or as either therapeutically or pharmaceutically active compound or in cosmetic applications. With the novel methods, the 6′sialylated disaccharide and/or 6′sialylated oligosaccharide can easily and effectively be provided, without the need for complicated, time and cost consuming synthetic processes.

For identification of the 6′sialylated disaccharide and/or 6′sialylated oligosaccharide of the disclosure produced in the cell as described herein, the monosaccharide or the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the 6′sialylated disaccharide and/or 6′sialylated oligosaccharide methods such as, e.g., acid-catalyzed hydrolysis, HPLC (high performance liquid chromatography) or GLC (gas-liquid chromatography) (after conversion to alditol acetates) may be used. To determine the glycosidic linkages, the 6′sialylated disaccharide and/or 6′sialylated oligosaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the 6′sialylated disaccharide and/or 6′sialylated oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

The separated and preferably also purified 6′sialylated disaccharide and/or 6′sialylated oligosaccharide as described herein is incorporated into a food (e.g., human food or feed), dietary supplement, pharmaceutical ingredient, cosmetic ingredient or medicine. In some embodiments, the 6′sialylated disaccharide and/or 6′sialylated oligosaccharide is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the 6′sialylated disaccharide and/or 6′sialylated oligosaccharide being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a 6′sialylated disaccharide and/or 6′sialylated oligosaccharide produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavorings.

In some embodiments, the 6′sialylated disaccharide and/or 6′sialylated oligosaccharide is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to roughly mimic human breast milk. In some embodiments, a 6′sialylated disaccharide and/or 6′sialylated oligosaccharide produced and/or purified by a process in this specification is included in infant formula to provide nutritional benefits similar to those provided by the oligosaccharides in human breast milk. In some embodiments, the 6′sialylated disaccharide and/or 6′sialylated oligosaccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils-such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the 6′sialylated disaccharide and/or 6′sialylated oligosaccharide in the infant formula is approximately the same concentration as the concentration of the 6′sialylated disaccharide and/or 6′sialylated oligosaccharide generally present in human breast milk.

In some embodiments, the 6′sialylated disaccharide and/or 6′sialylated oligosaccharide is incorporated into a feed preparation, wherein the feed is chosen from the list comprising pet food, animal milk replacer, veterinary product, post weaning feed, or creep feed.

As will be shown in the examples herein, the newly identified alpha-2,6-sialyltransferase have proven to be useful in the enzymatic and cell-based production of sialylated di- and/or oligosaccharides, preferably a 6′sialylated disaccharide and/or 6′sialylated oligosaccharide, more preferably 6′sialyllactose. The method and the cell of the disclosure preferably provide at least one of the following further surprising advantages when using the alpha-2,6-sialyltransferase as defined herein:
Higher titers of the sialylated oligosaccharide (g/L),
Higher purity of the sialylated oligosaccharide (g/L),
Higher production rate r (g sialylated oligosaccharide/L/h),
Higher cell performance index CPI (g sialylated oligosaccharide/g X),
Higher specific productivity Qp (g sialylated oligosaccharide/g X/h),
Higher yield on the carbon source used Y (g sialylated oligosaccharide/g carbon source used),
Higher yield on sucrose Ys (g sialylated oligosaccharide/g sucrose),
Higher uptake/conversion rate of the carbon source used Q (g carbon source/g X/h),
Higher sucrose uptake/conversion rate Qs (g sucrose/g X/h),
Higher lactose conversion/consumption rate rs (g lactose/h),
Higher secretion or excretion or extracellular transport of the sialylated oligosaccharide, and/or
Higher growth speed of the production host, when compared to an enzymatic production or a cell-based production using an identical enzymatic or genetic background but lacking the use of the newly identified alpha-2,6-sialyltransferase.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features that are still to be explained below can be used not only in the respectively specified combinations, but also in other combinations or on their own, without departing from the scope of the disclosure.

Moreover, the disclosure relates to the following specific embodiments:

1. An alpha-2,6-sialyltransferase for use in the production of a sialylated oligosaccharide, preferably a 6′sialylated oligosaccharide, more preferably 6′sialyllactose, wherein the alpha-2,6-sialyltransferase comprises an amino acid sequence that is at least to 80.0% identical over a stretch of at least 200 amino acids to the amino acid sequence as represented by SEQ ID NO: 1, preferably, the alpha-2,6-sialyltransferase is a lactose-accepting alpha-2,6-sialyltransferase.

2. The alpha-2,6-sialyltransferase according to embodiment 1, wherein the alpha-2,6-sialyltransferase comprises an amino acid sequence that is at least 80.0%, at least 85.0%, at least 90.0%, at least 95.0%, at least 96.0%, at least 97.0%, at least 98.0%, at least 98.5%, or at least 99% identical to the amino acid sequence as represented by SEQ ID NO: 1 over a stretch of at least 210, at least 220, at least 230, at least 240, at least 250, at least 260, at least 270, at least 280, at least 290 or at least 300 amino acids.

3. The alpha-2,6-sialyltransferase according to any one of embodiments 1 or 2, wherein the alpha-2,6-sialyltransferase comprises an amino acid sequence is at least 80.0%, at least 85.0%, at least 90.0%, at least 95.0%, at least 96.0%, at least 97.0%, at least 98.0%, at least 98.5%, or at least 99% identical to the full-length amino acid sequence as represented by SEQ ID NO: 1.

4. The alpha-2,6-sialyltransferase according to any one of embodiments 1 to 3, wherein the alpha-2,6-sialyltransferase comprises an amino acid sequence as represented by SEQ ID NO: 1.

5. Method for the production of a sialylated oligosaccharide, preferably a 6′sialylated oligosaccharide, more preferably 6′sialyllactose, the method comprising: contacting the isolated sialyltransferase of any one of embodiments 1 to 4 with a mixture comprising a donor substrate comprising a sialic acid residue, and an acceptor substrate chosen from the list comprising an oligosaccharide or a disaccharide, under conditions where the sialyltransferase catalyzes the transfer of a sialic acid residue from the donor substrate to the acceptor substrate, thereby producing the sialylated oligosaccharide.

6. Method according to embodiment 5, wherein the sialylated oligosaccharide is 6′sialyllactose and the acceptor substrate is a disaccharide.

7. Method for the production of a sialylated oligosaccharide, preferably a 6′sialylated oligosaccharide, more preferably 6′sialyllactose, the method comprising the steps of:
a) Providing
   i. CMP-sialic acid
   ii. An acceptor substrate, preferably lactose
   iii. A sialyltransferase, wherein the sialyltransferase is an alpha-2,6-sialyltransferase, which comprises an amino acid sequence that is at least to 80.0% identical over a stretch of at least 200 amino acids to the amino acid sequence as represented by SEQ ID NO: 1.
b) contacting the sialyltransferase and CMP-sialic acid with the acceptor substrate, preferably lactose, under conditions where the sialyltransferase catalyzes the transfer of a sialic acid residue from the CMP-sialic acid to the acceptor substrate resulting in the production of a sialylated oligosaccharide, preferably a 6′sialylated oligosaccharide, more preferably 6′sialyllactose,
c) preferably, separating the produced sialylated oligosaccharide.

8. Method according to embodiment 7, wherein the sialyltransferase is any one of the sialyltransferase of any one of embodiments 1 to 4.

9. Method according to any one of embodiments 5 to 8, the method comprising: contacting a cell extract comprising a sialyltransferase of any one of embodiments 1 to 4, with a mixture comprising a donor substrate comprising a sialic acid residue, and an acceptor substrate comprising an oligosaccharide or disaccharide, under conditions where the sialyltransferase catalyzes the transfer of a sialic acid residue from the donor substrate to the acceptor substrate, thereby producing the sialylated oligosaccharide.

10. Method according to any one of embodiments 5 to 8, wherein the sialylated oligosaccharide, preferably a 6′sialylated oligosaccharide, more preferably 6′sialyllactose, is produced in a cell-free system.

11. Method according to any one of embodiments 5 to 8, wherein the sialylated oligosaccharide is a 6′sialylated oligosaccharide and the 6′sialylated oligosaccharide is produced by a cell, preferably a single cell, the cell expressing an alpha-2,6-sialyltransferase, which comprises an amino acid sequence that is at least to 80.0% identical over a stretch of at least 200 amino acids to the amino acid sequence as represented by SEQ ID NO: 1.

12. Method according to embodiment 11, wherein the cell is a metabolically engineered cell, preferably wherein the cell is metabolically engineered for the production of the 6′sialylated oligosaccharide.

13. Method according to embodiment 11 or 12, wherein the method comprises the steps of:
i. providing a cell expressing, preferably heterologously expressing, more preferably heterologously overexpressing, the sialyltransferase,
ii. providing CMP-sialic acid, optionally, the CMP-sialic acid is produced by the cell, and
iii. providing an oligosaccharide or disaccharide, optionally, the oligosaccharide or disaccharide is produced by the cell, and
iv. cultivating and/or incubating the cell under conditions permissive to express the sialyltransferase, optionally permissive to produce the CMP-sialic acid and/or the oligosaccharide or disaccharide,
v. preferably, separating the 6′sialylated oligosaccharide from the cultivation.

14. The method according to embodiment 13, wherein the cultivation or incubating medium contains at least one carbon source selected from the group consisting of glucose, fructose, sucrose, and glycerol.

15. The method according to embodiment 13 or 14, wherein the cultivation or incubating medium contains at least one compound selected from the group consisting of lactose, galactose and sialic acid.

16. The method according to any one of embodiments 13 to 15, wherein the 6′sialylated oligosaccharide, is recovered from the cultivation or incubating medium and/or the cell.

17. Method according to any one of embodiments 11 to 16, the method comprising at least one of the following steps:
i) Use of a cultivation or incubation medium comprising at least 50, more preferably at least 75, more preferably at least 100, more preferably at least 120, more preferably at least 150 grams of precursor per liter of initial reactor volume wherein the reactor volume ranges from 250 mL to 10,000 m³ (cubic meter);
ii) Adding to the cultivation or incubation medium a precursor feed comprising at least 50, more preferably at least 75, more preferably at least 100, more preferably at least 120, more preferably at least 150 gram of precursor per liter of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than two-fold of the volume of the culture medium before the addition of the precursor feed;
iii) Adding to the cultivation or incubation medium a precursor feed comprising at least 50, more preferably at least 75, more preferably at least 100, more preferably at least 120, more preferably at least 150 gram of precursor per liter of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than two-fold of the volume of the culture medium before the addition of the precursor feed and wherein preferably the pH of the precursor feed is set between 3.0 and 7.0 and wherein preferably the temperature of the precursor feed is kept between 20° C. and 80° C.;
iv) Adding a precursor feed in a continuous manner to the cultivation or incubation medium over the course of 1 day, 2 days, 3 days, 4 days, 5 days by means of a feeding solution;
v) Adding a precursor feed in a continuous manner to the cultivation or incubation medium over the course of 1 day, 2 days, 3 days, 4 days, 5 days by means of a feeding solution and wherein the concentration of the precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the feeding solution is set between 3.0 and 7.0 and wherein preferably the temperature of the feeding solution is kept between 20° C. and 80° C.;
the method resulting in the 6′sialylated oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation.

18. Method according to any one of embodiments 11 to 17 for the production of the 6′sialylated oligosaccharide 6′sialyllactose, the method comprising at least one of the following steps:
i) Use of a cultivation or incubation medium comprising at least 50, more preferably at least 75, more preferably at least 100, more preferably at least 120, more preferably at least 150 grams of lactose per liter of initial reactor volume wherein the reactor volume ranges from 250 mL to 10,000 m³ (cubic meter);
ii) Adding to the cultivation or incubation medium a lactose feed comprising at least 50, more preferably at least 75, more preferably at least 100, more preferably at least 120, more preferably at least 150 gram of lactose per liter of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than two-fold of the volume of the culture medium before the addition of the lactose feed;
iii) Adding to the cultivation or incubation medium a lactose feed comprising at least 50, more preferably at least 75, more preferably at least 100, more preferably at least 120, more preferably at least 150 gram of lactose per liter of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than two-fold of the volume of the culture medium before the addition of the lactose feed and wherein preferably the pH of the lactose feed is set between 3.0 and 7.0 and wherein preferably the temperature of the lactose feed is kept between 20° C. and 80° C.;
iv) Adding a lactose feed in a continuous manner to the cultivation or incubation medium over the course of 1 day, 2 days, 3 days, 4 days, 5 days by means of a feeding solution;
v) Adding a lactose feed in a continuous manner to the cultivation or incubation medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the feeding solution is set between 3.0 and 7.0 and wherein preferably the temperature of the feeding solution is kept between 20° C. and 80° C.;
the method resulting in the 6′sialyllactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation.

19. A metabolically engineered cell for the production of a 6′sialylated oligosaccharide, preferably 6′sialyllactose, wherein the cell has been metabolically engineered to possess an alpha-2,6-sialyltransferase, which comprises an amino acid sequence that is at least to 80.0% identical over a stretch of at least 200 amino acids to the amino acid sequence as represented by SEQ ID NO: 1, preferably the alpha-2,6-sialyltransferase is a lactose-accepting alpha-2,6-sialyltransferase.

20. The cell according to embodiment 19, wherein the sialyltransferase is any one of the sialyltransferases of embodiments 1 to 4.

21. The cell according to any one of embodiments 19 to 20, wherein the cell contains a nucleic acid molecule, which comprises a nucleotide sequence that encodes any one of the sialyltransferases of embodiments 1 to 4.

22. The cell according to any one of embodiments 19 to 21, wherein the cell is a bacterium, fungus, yeast, a plant cell, an animal cell, or a protozoan cell,
preferably, the bacterium is an Escherichia coli strain, more preferably an Escherichia coli strain, which is a K-12 strain, even more preferably, the Escherichia coli K-12 strain is E. coli MG1655,
preferably, the fungus belongs to a genus chosen from the group comprising Rhizopus, Dictyostelium, Penicillium, Mucor or Aspergillus,
preferably, the yeast belongs to a genus chosen from the group comprising Saccharomyces, Zygosaccharomyces, Pichia, Komagataella, Hansemila, Yarrowia, Starmerella, Kluyveromyces or Debaromyces,
preferably, the plant cell is an algal cell or is derived from tobacco, alfalfa, rice, tomato, cotton, rapeseed, soy, maize, or corn plant,
preferably, the animal cell is derived from non-human mammals, birds, fish, invertebrates, reptiles, amphibians or insects or is a genetically modified cell line derived from human cells excluding embryonic stem cells, more preferably, the human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably, the insect cell is derived from Spodoptera frugiperda, Bombyx mori, Mamestra brassicae, Trichoplusia ni or Drosophila melanogaster,
preferably, the protozoan cell is a Leishmania tarentolae cell.

23. The cell according to any one of embodiments 19 to 22, wherein the cell is selected from the group consisting of prokaryotic cells and eukaryotic cells, preferably from the group consisting of yeast cells, bacterial cells, archaebacterial cells, algae cells, and fungal cells.

24. Cell according to any one embodiments 19 to 23, the cell comprising i) a sequence comprising a polynucleotide encoding the alpha-2,6-sialyltransferase, wherein the sequence is a sequence foreign to the cell and wherein the sequence is integrated in the genome of the cell, or ii) containing a vector comprising a polynucleotide encoding the alpha-2,6-sialyltransferase, wherein the polynucleotide being operably linked to control sequences recognized by a cell transformed with the vector.

25. Cell according to any one of embodiments 19 to 24, wherein the cell comprises a catabolic pathway for selected mono-, di- or oligosaccharides, which is at least partially inactivated, the mono-, di-, or oligosaccharides being involved in and/or required for the synthesis of a 6′sialylated oligosaccharide, preferably 6′sialyllactose.

26. An isolated nucleic acid molecule encoding an alpha-2,6-sialyltransferase of any one of embodiments 1 to 4.

27. A vector comprising the nucleic acid molecule of embodiment 26.

28. Use of an alpha-2,6-sialyltransferase of any one of embodiments 1 to 4 for production of a 6′sialylated oligosaccharide, preferably 6′sialyllactose.

29. Use of a cell according to any one of embodiments 19 to 25 for production of a 6′sialylated oligosaccharide, preferably 6′sialyllactose.

30. Use of a method according to any one of embodiments 5 to 18 for production of a 6′sialylated oligosaccharide, preferably 6′sialyllactose.

31. Use of an isolated nucleic acid molecule according to embodiment 26 for production of a 6′sialylated oligosaccharide, preferably 6′sialyllactose.

32. Use of a vector according to embodiment 26 for production of a 6′sialylated oligosaccharide, preferably 6′sialyllactose.

Moreover, the disclosure relates to the following preferred specific embodiments:

1. An alpha-2,6-sialyltransferase for use in the production of a 6′sialylated disaccharide and/or 6′sialylated oligosaccharide, preferably 6′sialyllactose, wherein the alpha-2,6-sialyltransferase comprises an amino acid sequence that is at least 80.0% identical over a stretch of at least 200 amino acids to the amino acid sequence as represented by SEQ ID NO: 1, preferably, the alpha-2,6-sialyltransferase is a lactose-accepting alpha-2,6-sialyltransferase.

2. The alpha-2,6-sialyltransferase according to preferred embodiment 1, wherein the alpha-2,6-sialyltransferase comprises an amino acid sequence that is at least 80.0%, at least 85.0%, at least 90.0%, at least 95.0%, at least 96.0%, at least 97.0%, at least 98.0%, at least 98.5%, or at least 99% identical to the amino acid sequence as represented by SEQ ID NO: 1 over a stretch of at least 210, at least 220, at least 230, at least 240, at least 250, at least 260, at least 270, at least 280, at least 290 or at least 300 amino acids.

3. The alpha-2,6-sialyltransferase according to any one of preferred embodiments 1 or 2, wherein the alpha-2,6-sialyltransferase comprises an amino acid sequence that is at least 80.0%, at least 85.0%, at least 90.0%, at least 95.0%, at least 96.0%, at least 97.0%, at least 98.0%, at least 98.5%, or at least 99% identical to the full-length amino acid sequence as represented by SEQ ID NO: 1.

4. The alpha-2,6-sialyltransferase according to any one of preferred embodiments 1 to 3, wherein the alpha-2,6-sialyltransferase comprises an amino acid sequence as represented by SEQ ID NO: 1.

5. Method for the production of a 6′sialylated disaccharide and/or 6′sialylated oligosaccharide, preferably 6′sialyllactose, the method comprising: contacting an alpha-2,6-sialyltransferase of any one of preferred embodiments 1 to 4 with a mixture comprising a donor comprising a sialic acid residue, and an acceptor chosen from the list comprising an oligosaccharide or a disaccharide, under conditions wherein the alpha-2,6-sialyltransferase catalyzes the transfer of a sialic acid residue from the donor to the acceptor, thereby producing the sialylated di- and/or oligosaccharide.

6. Method according to preferred embodiment 5, wherein the 6′sialylated oligosaccharide is 6′sialyllactose and the acceptor is a disaccharide.

7. Method for the production of a 6′sialylated disaccharide and/or 6′sialylated oligosaccharide, preferably 6′sialyllactose, the method comprising the steps of:
a) Providing
   i. CMP-sialic acid,
   ii. an acceptor, preferably lactose, and
   iii. a sialyltransferase, wherein the sialyltransferase is an alpha-2,6-sialyltransferase of any one of preferred embodiments 1 to 4,
b) contacting the sialyltransferase and CMP-sialic acid with the acceptor, preferably lactose, under conditions wherein the sialyltransferase catalyzes the transfer of a sialic acid residue from the CMP-sialic acid to the acceptor resulting in the production of 6′sialylated disaccharide and/or 6′sialylated oligosaccharide, preferably 6′sialyllactose,
c) preferably, separating the produced sialylated di- and/or oligosaccharide,
d) optionally, recovering the 6′sialylated disaccharide and/or 6′sialylated oligosaccharide, preferably 6′sialyllactose, from the cultivation or incubation medium and/or the cell.

8. Method according to any one of preferred embodiment 5 to 7, the method comprising: contacting a cell extract comprising an alpha-2,6-sialyltransferase of any one of preferred embodiments 1 to 4, with a mixture comprising a donor comprising a sialic acid residue, and an acceptor comprising an oligosaccharide or disaccharide, under conditions wherein the alpha-2,6-sialyltransferase catalyzes the transfer of a sialic acid residue from the donor to the acceptor, thereby producing the 6′sialylated disaccharide and/or 6′sialylated oligosaccharide, preferably 6′sialyllactose.

9. Method according to any one of preferred embodiments 5 to 8, wherein the 6′sialylated disaccharide and/or 6′sialylated oligosaccharide, preferably 6′sialyllactose, is produced in a cell-free system.

10. Method for the production of a 6′sialylated disaccharide and/or 6′sialylated oligosaccharide, preferably 6′sialyllactose, the method comprising the steps of:
i. providing a cell, preferably a single cell, expressing, preferably heterologously expressing, more preferably overexpressing, even more preferably heterologously overexpressing, an alpha-2,6-sialyltransferase of any one of preferred embodiments 1 to 4,
ii. providing CMP-sialic acid, optionally, the CMP-sialic acid is produced by the cell, and
iii. providing an oligosaccharide or disaccharide, optionally, the oligosaccharide or disaccharide is produced by the cell, and
iv. cultivating and/or incubating the cell under conditions permissive to express the sialyltransferase, optionally permissive to produce the CMP-sialic acid and/or the oligosaccharide or disaccharide,
v. preferably, separating the 6′sialylated disaccharide and/or 6′sialylated oligosaccharide, preferably 6′sialyllactose, from the cultivation or incubation.
vi. optionally, recovering the 6′sialylated disaccharide and/or 6′sialylated oligosaccharide, preferably 6′sialyllactose, from the cultivation or incubation medium and/or the cell.

11. Method according to preferred embodiment 10, wherein the cell is a metabolically engineered cell, preferably wherein the cell is metabolically engineered for the production of the 6′sialylated disaccharide and/or 6′sialylated oligosaccharide, preferably 6′sialyllactose.

12. The method according to any one of preferred embodiments 10 or 11, wherein the cultivation medium contains i) at least one carbon source selected from the group consisting of glucose, fructose, sucrose, and glycerol, and/or ii) at least one compound selected from the group consisting of lactose, galactose, sialic acid, UDP-galactose (UDP-Gal) and CMP-sialic acid.

13. Method according to any one of preferred embodiments 5 to 12, the method comprising:
i. Use of a cultivation or incubation medium comprising at least one precursor and/or acceptor for the production of the 6′sialylated disaccharide and/or 6′sialylated oligosaccharide, preferably 6′sialyllactose, and/or
ii. Adding to the cultivation or incubation medium at least one precursor and/or acceptor feed for the production of the 6′sialylated disaccharide and/or 6′sialylated oligosaccharide, preferably 6′sialyllactose,
preferably, the precursor is chosen from the list comprising sialic acid, CMP-sialic acid, glucose, galactose and UDP-galactose,
preferably, the acceptor is lactose.

14. Method according to any one of preferred embodiments 5 to 13, the method comprising at least one of the following steps:
i. Use of a cultivation or incubation medium comprising at least one precursor and/or acceptor;
ii. Adding to the cultivation or incubation medium in a reactor or incubator at least one precursor and/or acceptor feed wherein the total reactor or incubator volume ranges from 250 mL to 10,000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the precursor and/or acceptor feed;
iii. Adding to the cultivation or incubation medium in a reactor or incubator at least one precursor and/or acceptor feed wherein the total reactor or incubator volume ranges from 250 mL to 10,000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the precursor and/or acceptor feed and wherein preferably, the pH of the precursor and/or acceptor feed is set between 2.0 and 10.0, preferably between 3.0 and 7.0, and wherein preferably, the temperature of the precursor and/or acceptor feed is kept between 20° C. and 80° C.;
iv. Adding at least one precursor and/or acceptor feed in a continuous manner to the cultivation or incubation medium over the course of 1 day, 2 days, 3 days, 4 days, 5 days by means of a precursor and/or acceptor feeding solution;
v. Adding at least one precursor and/or acceptor feed in a continuous manner to the cultivation or incubation medium over the course of 1 day, 2 days, 3 days, 4 days, 5 days by means of a precursor and/or acceptor feeding solution and wherein the concentration of the precursor and/or acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the precursor and/or acceptor feeding solution is set between 2.0 and 10.0, preferably between 3.0 and 7.0, and wherein preferably, the temperature of the precursor and/or acceptor feeding solution is kept between 20° C. and 80° C.;
the method resulting in the 6′sialylated disaccharide and/or 6′sialylated oligosaccharide, preferably 6′sialyllactose, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation,
wherein, preferably, the precursor is chosen from the list comprising sialic acid, CMP-sialic acid, glucose, galactose and UDP-galactose and wherein, preferably, the acceptor is lactose.

15. Method according to any one of preferred embodiments 5 to 13, the method comprising at least one of the following steps:
i) Use of a cultivation or incubation medium comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10,000 m³ (cubic meter);
ii) Use of a cultivation or incubation medium comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10,000 m³ (cubic meter);
iii) Adding to the cultivation or incubation medium in a reactor or incubator a precursor feed comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10,000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the precursor feed
iv) Adding to the cultivation or incubation medium in a reactor or incubator an acceptor feed comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10,000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the acceptor feed;
v) Adding to the cultivation or incubation medium a precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10,000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the precursor feed and wherein preferably, the pH of the precursor feed is set between 2.0 and 10.0, preferably between 3.0 and 7.0, and wherein preferably, the temperature of the precursor feed is kept between 20° C. and 80° C.;
vi) Adding to the cultivation or incubation medium an acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10,000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the acceptor feed and wherein preferably, the pH of the acceptor feed is set between 2.0 and 10.0, preferably between 3.0 and 7.0, and wherein preferably, the temperature of the acceptor feed is kept between 20° C. and 80° C.;
vii) Adding a precursor and/or acceptor feed in a continuous manner to the cultivation or incubation medium over the course of 1 day, 2 days, 3 days, 4 days, 5 days by means of a precursor and/or acceptor feeding solution;
viii) Adding a precursor feed in a continuous manner to the cultivation or incubation medium over the course of 1 day, 2 days, 3 days, 4 days, 5 days by means of a precursor feeding solution and wherein the concentration of the precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the precursor feeding solution is set between 2.0 and 10.0, preferably between 3.0 and 7.0, and wherein preferably, the temperature of the precursor feeding solution is kept between 20° C. and 80° C.;
ix) Adding an acceptor feed in a continuous manner to the cultivation or incubation medium over the course of 1 day, 2 days, 3 days, 4 days, 5 days by means of an acceptor feeding solution and wherein the concentration of the acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the acceptor feeding solution is set between 2.0 and 10.0, preferably between 3.0 and 7.0, and wherein preferably, the temperature of the acceptor feeding solution is kept between 20° C. and 80° C.;
the method resulting in the 6′sialylated disaccharide and/or 6′sialylated oligosaccharide, preferably 6′sialyllactose, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation,
wherein preferably, the precursor is chosen from the list comprising sialic acid, CMP-sialic acid, glucose, galactose and UDP-galactose and wherein preferably, the acceptor is lactose.

16. A metabolically engineered cell for the production of a 6′sialylated disaccharide and/or 6′sialylated oligosaccharide, preferably 6′sialyllactose, wherein the cell has been metabolically engineered to possess, preferably to express, an alpha-2,6-sialyltransferase of any one of preferred embodiments 1 to 4.

17. Cell according to preferred embodiment 16, wherein the cell contains a nucleic acid molecule that comprises a nucleotide sequence that encodes any one of the alpha-2,6-sialyltransferases of preferred embodiments 1 to 4.

18. Cell according to preferred embodiment 16 or 17, wherein the cell is a bacterium, fungus, yeast, a plant cell, an animal cell, or a protozoan cell,
preferably, the bacterium belongs to a phylum chosen from the group comprising Proteobacteria, Firmicutes, Cyanobacteria, Deinococcus-Thermus and Actinobacteria; more preferably, the bacterium belongs to a family chosen from the group comprising Enterobacteriaceae, Bacillaceae, Lactobacillus, Corynebacteriaceae and Vibrionaceae; even more preferably, the bacterium is chosen from the list comprising an Escherichia coli strain, a Bacillus subtilis strain, a Vibrio natriegens strain; even more preferably, the Escherichia coli strain is a K-12 strain, most preferably, the Escherichia coli K-12 strain is E. coli MG1655,
preferably, the fungus belongs to a genus chosen from the group comprising Rhizopus, Dictyostelium, Penicillium, Mucor or Aspergillus,
preferably, the yeast belongs to a genus chosen from the group comprising Saccharomyces, Zygosaccharomyces, Pichia, Komagataella, Hansemila, Yarrowia, Starmerella, Kluyveromyces, Debaromyces, Candida, Schizosaccharomyces, Schwanniomyces or Torulaspora; more preferably, the yeast is selected from the group consisting of: Saccharomyces cerevisiae, Hansemila polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
preferably, the plant cell is an algal cell or is derived from tobacco, alfalfa, rice, tomato, cotton, rapeseed, soy, maize, or corn plant,
preferably, the animal cell is derived from insects, amphibians, reptiles, invertebrates, fish, birds or mammalian cells excluding human embryonic stem cells, more preferably, the mammalian cell is chosen from the list comprising an epithelial cell, an embryonic kidney cell, a fibroblast cell, a COS cell, a Chinese hamster ovary (CHO) cell, a murine myeloma cell, an NIH-3T3 cell, a lactocyte derived from mammalian-induced pluripotent stem cells, more preferably, the mammalian-induced pluripotent stem cells are human-induced pluripotent stem cells, a post-parturition mammary epithelium cell, a polarized mammary cell, more preferably, the polarized mammary cell is selected from the group comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof, more preferably, the insect cell is derived from Spodoptera frugiperda, Bombyx mori, Mamestra brassicae, Trichoplusia ni or Drosophila melanogaster,
preferably, the protozoan cell is a Leishmania tarentolae cell.

19. Cell according to any one of preferred embodiments 16 to 18, wherein the cell is selected from the group consisting of prokaryotic cells and eukaryotic cells, preferably from the group consisting of yeast cells, bacterial cells, archaebacterial cells, algae cells, and fungal cells.

20. Cell according to any one preferred embodiments 16 to 19, wherein the cell comprises a nucleic acid molecule comprising a polynucleotide sequence encoding the alpha-2,6-sialyltransferase of any one of preferred embodiments 1 to 4 and operably linked to control sequences recognized by the cell, wherein the sequence is foreign to the cell, the sequence further i) being integrated in the genome of the cell and/or ii) presented to the cell on a vector.

21. Cell according to any one of preferred embodiments 16 to 20, wherein the cell comprises a catabolic pathway for selected mono-, di- or oligosaccharides that is at least partially inactivated, the mono-, di-, or oligosaccharides being involved in and/or required for the synthesis of a sialylated di- and/or oligosaccharide, preferably a 6′sialylated disaccharide and/or 6′sialylated oligosaccharide, more preferably 6′sialyllactose.

22. Cell according to any one of preferred embodiments 16 to 21, wherein the cell produces the 6′sialylated disaccharide and/or 6′sialylated oligosaccharide intracellularly and wherein a fraction or substantially all of the produced 6′sialylated disaccharide and/or 6′sialylated oligosaccharide remains intracellularly and/or is excreted outside the cell via passive or active transport.

23. Cell according to any one of preferred embodiments 16 to 22, wherein the cell is further genetically modified for
i) modified expression of an endogenous membrane protein, and/or
ii) modified activity of an endogenous membrane protein, and/or
iii) expression of a homologous membrane protein, and/or
iv) expression of a heterologous membrane protein,
v) wherein the membrane protein is involved in the secretion of the 6′sialylated disaccharide and/or 6′sialylated oligosaccharide outside the cell.

24. Cell according to any one of preferred embodiments 16 to 23, wherein the cell is further genetically modified for
i) modified expression of an endogenous membrane protein, and/or
ii) modified activity of an endogenous membrane protein, and/or
iii) expression of a homologous membrane protein, and/or
iv) expression of a heterologous membrane protein,
v) wherein the membrane protein is involved in the uptake of a precursor and/or an acceptor for the synthesis of the 6′sialylated disaccharide and/or 6′sialylated oligosaccharide, preferably wherein the membrane protein is involved in the uptake of all of the required precursors, more preferably wherein the membrane protein is involved in the uptake of all of the acceptors.

25. Cell according to preferred embodiment 23 or 24, wherein the membrane protein is chosen from the list comprising porters, P—P-bond-hydrolysis-driven transporters, β-barrel porins, auxiliary transport proteins, putative transport proteins and phosphotransfer-driven group translocators,
preferably, the porters comprise MFS transporters, sugar efflux transporters and siderophore exporters,
preferably, the P—P-bond-hydrolysis-driven transporters comprise ABC transporters and siderophore exporters.

26. Cell according to any one of preferred embodiment 16 to 25, wherein the cell comprises a modification for reduced production of acetate compared to a non-modified progenitor.

27. Cell according to preferred embodiment 27, wherein the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose: undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIAGlc, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase compared to a non-modified progenitor.

28. Cell according to any one of preferred embodiment 16 to 27, wherein the cell is capable to produce phosphoenolpyruvate (PEP).

29. Cell according to any one of preferred embodiment 16 to 28, wherein the cell is modified for enhanced production and/or supply of phosphoenolpyruvate (PEP) compared to a non-modified progenitor.

30. An isolated nucleic acid molecule encoding an alpha-2,6-sialyltransferase of any one of preferred embodiments 1 to 4.

31. A vector comprising the nucleic acid molecule of preferred embodiment 30.

32. Use of an alpha-2,6-sialyltransferase of any one of preferred embodiments 1 to 4 for production of a sialylated di- and/or oligosaccharide, preferably a 6′sialylated disaccharide and/or 6′sialylated oligosaccharide, more preferably 6′sialyllactose.

33. Use of a cell according to any one of preferred embodiments 16 to 29 for production of a sialylated di- and/or oligosaccharide, preferably a 6′sialylated disaccharide and/or 6′sialylated oligosaccharide, more preferably 6′sialyllactose.

34. Use of a method according to any one of preferred embodiment 5 to 15 for production of a sialylated di- and/or oligosaccharide, preferably a 6′sialylated disaccharide and/or 6′sialylated oligosaccharide, more preferably 6′sialyllactose.

35. Use of an isolated nucleic acid molecule according to preferred embodiment 30 for production of a sialylated di- and/or oligosaccharide, preferably a 6′sialylated disaccharide and/or 6′sialylated oligosaccharide, more preferably 6′sialyllactose.

36. Use of a vector according to preferred embodiment 31 for production of a sialylated di- and/or oligosaccharide, preferably a 6′sialylated disaccharide and/or 6′sialylated oligosaccharide, more preferably 6′sialyllactose.

The disclosure will be described in more detail in the examples. The following examples will serve as further illustration and clarification of the disclosure and are not intended to be limiting.

### EXAMPLES

### Example 1. Materials and Methods—General

### Heterologous and Homologous Expression

Genes that needed to be expressed, be it from a plasmid or from the genome were synthetically synthetized with one of the following companies: IDT or Twist Bioscience. Proteins described in the disclosure are summarized in Tables 1 and 2. Unless stated otherwise, the UniProt IDs of the proteins described correspond to their sequence version 01 as present in the UniProt Database version release 2021_03 of 9 Jun. 2021. Expression could be further facilitated by optimizing the codon usage to the codon usage of the expression host. Genes were optimized using the tools of the supplier.

TABLE 1
Protein with corresponding SEQ ID
NO as described in the disclosure:

| | | | Country of origin of |
|---|---|---|---|
| SEQ ID | | | digital sequence |
| NO: | Organism | Origin | information |
| 1 | Simonsiella muelleri ATCC | Synthetic | Austria |
| | 29453 | | |

TABLE 2
Overview of proteins with corresponding UniProt IDs (sequence version 01,
UniProt Database 2021_03 of 9 Jun. 2021) as described in the disclosure:

| | | | | | Country of |
|---|---|---|---|---|---|
| SEQ | | | | | origin of digital |
| ID | | | | | sequence |
| NO: | UniProt ID | Name | Organism | Origin | information |
| 6 | A7LVG6 | AGE | Bacteroides ovatus | Synthetic | USA |
| 3 | A0ZZH6 | BaSP | Bifidobacterium | Synthetic | Germany |
| | | | adolescentis | | |
| | Q8NFW8 | CMAS | Homo sapiens | Synthetic | Unknown |
| 17 | E0IXR1 | cscB | Escherichia coli W | Synthetic | USA |
| | Q61420 | CST | Mus musculus | Synthetic | USA |
| 2 | Q03417 | Frk | Zymomonas mobilis | Synthetic | United Kingdom |
| 14 | P31120* | glmM | Escherichia coli K- | Synthetic | USA |
| | | | 12 MG1655 | | |
| 8 | P17169** | glmS | Escherichia coli K- | Synthetic | USA |
| | | | 12 MG1655 | | |
| | P0CI73 | glmS | Bacillus subtilis | Synthetic | USA |
| | Q8NND3*** | glmS | Corynebacterium | Synthetic | USA |
| | | | glutamicum | | |
| 15 | P0ACC7 | glmU | Escherichia coli K- | Synthetic | USA |
| | | | 12 MG1655 | | |
| | Q06210 | GFPT1 | Homo sapiens | Synthetic | Unknown |
| | Q96EK6 | GNA1 | Homo sapiens | Synthetic | Unknown |
| 5 | P43577 | GNA1 | Saccharomyces | Synthetic | USA |
| | | | cerevisiae | | |
| | Q9Y223 | GNE | Homo sapiens | Synthetic | Unknown |
| | Q91WG8 | GNE | Mus musculus | Synthetic | USA |
| 13 | P07921 | LAC12 | Kluyveromyces | Synthetic | USA |
| | | | lactis | | |
| 4 | P02920 | LacY | Escherichia coli K- | Synthetic | USA |
| | | | 12 MG1655 | | |
| | Q9NR45 | NANS | Homo sapiens | Synthetic | Unknown |
| | Q99KK2 | NeuA | Mus musculus | Synthetic | USA |
| 11 | A0A849CI62 | NeuA | Pasteurella | Synthetic | USA |
| | | | multocida | | |
| 16 | Q93MP9 | NeuB | Campylobacter | Synthetic | USA |
| | | | jejuni | | |
| 7 | E0NCD4 | NeuB | Neisseria | Synthetic | United Kingdom |
| | | | meningitidis | | |
| 10 | Q93MP8 | NeuC | Campylobacter | Synthetic | USA |
| | | | jejuni | | |
| 12 | O66375 | PdST6 | Photobacterium | Synthetic | Japan |
| | | | damselae | | |
| | O95394 | PGM3 | Homo sapiens | Synthetic | Unknown |
| 18 | A8QYL1 | P-JT-ISH-224- | Photobacterium sp. | Synthetic | Japan |
| | | ST6 | JT-ISH-224 | | |
| | P0A840 | SurE | Escherichia coli K- | Synthetic | USA |
| | | | 12 MG1655 | | |
| | Q16222 | UAP1 | Homo sapiens | Synthetic | Unknown |
| 9 | P77475 | yqaB | Escherichia coli K- | Synthetic | USA |
| | | | 12 MG1655 | | |

*Sequence version 03 (23 Jan. 2007) as present in the UniProt Database 2021_03 of 9 June 2021
**Sequence version 04 (23 Jan. 2007) as present in the UniProt Database 2021_03 of 9 June 2021
***Sequence version 02 (23 Jan. 2007) as present in the UniProt Database 2021_03 of 9 June 2021
**** Sequence version 02 (1 Dec. 2000) as present in the UniProt Database 2021_03 of 9 June
2021

### Calculation of Percentage Identity Between Nucleotide or Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48:443-453) to find the global (i.e., spanning the full-length sequences) alignment of two sequences that maximizes the number of matches and minimizes the number of gaps. The BLAST algorithm (Altschul et al., J. Mol. Biol. (1990) 215:403-10) calculates the global percentage sequence identity (i.e., over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity (i.e., spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T. F., Waterman M. S. (1981) J. Mol. Biol. 147 (1); 195-7).

### Analytical Analysis

Standards such as but not limited to sucrose, lactose, LacNAc, lacto-N-biose (LNB), fucosylated LacNAc (2′FLacNAc, 3-FLacNAc), sialylated LacNAc, (3′SLacNAc, 6′SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/minute. The ELS detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/minute. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/minute. The temperature of the RI detector was set at 35° C.

For analysis on a mass spectrometer, a Waters Xevo TQ-MS with Electron Spray Ionization (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 minutes using the following gradient: an initial increase from 2 to 12% of eluent B over 21 minutes, a second increase from 12 to 40% of eluent B over 11 minutes and a third increase from 40 to 100% of eluent B over 5 minutes. As a washing step 100% of eluent B was used for 5 minutes. For column equilibration, the initial condition of 2% of eluent B was restored in 1 minute and maintained for 12 minutes.

Both neutral and sialylated sugars at low concentrations (below 50 mg/L) were analyzed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 minutes while maintaining a constant ratio of 25% of eluent B using the following gradient: an initial isocratic step maintained for 10 minutes of 75% of eluent A, an initial increase from 0 to 4% of eluent C over 8 minutes, a second isocratic step maintained for 6 minutes of 71% of eluent A and 4% of eluent C, a second increase from 4 to 12% of eluent C over 2.6 minutes, a third isocratic step maintained for 3.4 minutes of 63% of eluent A and 12% of eluent C and a third increase from 12 to 48% of eluent C over 5 minutes. As a washing step 48% of eluent C was used for 3 minutes. For column equilibration, the initial condition of 75% of eluent A and 0% of eluent C was restored in 1 minute and maintained for 11 minutes. The applied flow was 0.5 mL/minute.

### Example 2. Materials and Methods—Escherichia coli

### Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4·7H2O, 30 g/L sucrose or 30 g/L glycerol, 1 ml/L vitamin solution, 100 μl/L molybdate solution, and 1 mL/L selenium solution. As specified in the respective examples, 0.30 g/L sialic acid, 20 g/L lactose, 20 g/L LacNAc and/or 20 g/L LNB were additionally added to the medium as precursor(s). The minimal medium was set to a pH of 7 with 1M KOH. Vitamin solution consisted of 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3BO4, 0.4 g/L Na2EDTA·2H2O and 1.01 g/L thiamine.HCl. The molybdate solution contained 0.967 g/L NaMoO4·2H2O. The selenium solution contained 42 g/L Seo2.

The minimal medium for fermentations contained 6.75 g/L NH4Cl, 1.25 g/L (NH4) 2SO4, 2.93 g/L KH2PO4 and 7.31 g/L KH2PO4, 0.5 g/L NaCl, 0.5 g/L MgSO4·7H2O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose, 20 g/L LacNAc and/or 20 g/L LNB were additionally added to the medium as precursor(s).

Complex medium was sterilized by autoclaving (121° C., 21 minutes) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F, phi80dlacZΔM15, Δ(lacZYA-argF) U169, deoR, recA1, endA1, hsdR17 (rk⁻, mk⁺), phoA, supE44, lambda⁻, thi-1, gyrA96, relA1) bought from Invitrogen.

### Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645).

In an example for sialic acid production, the mutant strain was derived from E. coli K12 MG1655 comprising knock-outs of the E. coli nagA, nagB, poxB, ackA, pta, IdhA, nanA, nanE, nanK genes and genomic knock-ins of constitutive transcriptional units containing a glucosamine 6-phosphate N-acetyltransferase like, e.g., GNA1 from Saccharomyces cerevisiae with UniProt ID P43577 (SEQ ID NO: 5), an N-acetylglucosamine 2-epimerase like, e.g., AGE from Bacteroides ovatus with UniProt ID A7LVG6 (SEQ ID NO: 6) and an N-acetylneuraminate (Neu5Ac) synthase like, e.g., NeuB from Neisseria meningitidis with UniProt ID E0NCD4 (SEQ ID NO: 7) or an N-acetylneuraminate (Neu5Ac) synthase like, e.g., NeuB from Campylobacter jejuni with UniProt ID Q93MP9 (SEQ ID NO: 16). Sialic acid production was further optimized in the mutant E. coli strain with a genomic knock-in of a constitutive transcriptional unit comprising a mutated variant of the L-glutamine-D-fructose-6-phosphate aminotransferase (glmS*54) from E. coli with SEQ ID NO: 8 (differing from the wild-type E. coli glmS protein with UniProt ID P17169 (Sequence version 04, 23 Jan. 2007) by an A39T, an R250C and an G472S mutation). Sialic acid production can also be obtained by knock-outs of the E. coli nagA, nagB, poxB, ackA, pta, IdhA, nanA, nanE, nanK genes and genomic knock-ins of constitutive transcriptional units containing a phosphoglucosamine mutase like, e.g., glmM from E. coli with UniProt ID P31120 (Sequence version 03, 23 Jan. 2007) (SEQ ID NO: 14), an N-acetylglucosamine-1-phosphate uridyltransferase/glucosamine-1-phosphate acetyltransferase like, e.g., glmU from E. coli with UniProt ID P0ACC7 (SEQ ID NO: 15), a UDP-N-acetylglucosamine 2-epimerase like, e.g., NeuC from Campylobacter jejuni with UniProt ID Q93MP8 (SEQ ID NO: 10) and an N-acetylneuraminate synthase like, e.g., NeuB from N. meningitidis with UniProt ID E0NCD4 (SEQ ID NO: 7). Also in this mutant strain, sialic acid production can further be optimized with a genomic knock-in of constitutive transcriptional units comprising the mutant glmS*54 from E. coli differing from the wild-type E. coli glmS protein with UniProt ID P17169 (Sequence version 04, 23 Jan. 2007) by an A39T, an R250C and an G472S mutation as described by Deng et al. (Biochimie 88, 419-29 (2006) (SEQ ID NO: 8). For 6′SL or sialylated oligosaccharide production, the sialic acid production strains further need to express an N-acylneuraminate cytidylyltransferase like, e.g., NeuA from Pasteurella multocida with UniProt ID A0A849CI62 (SEQ ID NO: 11), and a beta-galactoside alpha-2,6-sialyltransferase. Constitutive transcriptional units of PmNeuA and sialyltransferases can be delivered to the mutant strain either via genomic knock-in or via expression plasmids. If the mutant strains producing sialic acid and CMP-sialic acid were intended to make sialylated lactose structures, and for 6′SL production, the strains were additionally modified with genomic knock-outs of the E. coli LacZ, LacY and LacA genes and with a genomic knock-in of a constitutive transcriptional unit for a lactose permease like, e.g., the E. coli LacY with UniProt ID P02920 (SEQ ID NO: 4). All mutant strains producing sialic acid, CMP-sialic acid and/or sialylated oligosaccharides could optionally be adapted for growth on sucrose via genomic knock-ins of constitutive transcriptional units containing a sucrose transporter like, e.g., CscB from E. coli W with UniProt ID E0IXR1 (SEQ ID NO: 17), a fructose kinase like, e.g., Frk originating from Z. mobilis with UniProt ID Q03417 (SEQ ID NO: 2) and a sucrose phosphorylase like, e.g., BaSP originating from B. adolescentis with UniProt ID A0ZZH6 (SEQ ID NO: 3).

Preferably but not necessarily, the glycosyltransferases, the proteins involved in nucleotide-activated sugar synthesis and/or the membrane transporter proteins were N- and/or C-terminally fused to a solubility enhancer tag like, e.g., a SUMO-tag, an MBP-tag, His, FLAG, Strep-II, Halo-tag, NusA, thioredoxin, GST and/or the Fh8-tag to enhance their solubility (Costa et al., Front. Microbiol. 2014, doi.org/10.3389/fmicb.2014.00063; Fox et al., Protein Sci. 2001, 10 (3), 622-630; Jia and Jeaon, Open Biol. 2016, 6:160196).

Optionally, the mutant E. coli strains were modified with a genomic knock-ins of a constitutive transcriptional unit encoding a chaperone protein like, e.g., DnaK, DnaJ, GrpE or the GroEL/ES chaperonin system (Baneyx F., Palumbo J. L. (2003) Improving Heterologous Protein Folding via Molecular Chaperone and Foldase Co-Expression. In: Vaillancourt P.E. (eds) E. coli Gene Expression Protocols. Methods in Molecular Biology™, vol 205. Humana Press).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, glgA, glgB, malQ, otsA and yaiP.

All constitutive promoters, UTRs and terminator sequences originated from the libraries described by Mutalik et al. (Nat. Methods 2013, No. 10, 354-360) and Cambray et al. (Nucleic Acids Res. 2013, 41 (9), 5139-5148): the genes were expressed using promoters MutalikP5 (“PROM0005_MutalikP5”) and apFAB82 (“PROM0050_apFAB82”) as described by Mutalik et al. (Nat. Methods 2013, No. 10, 354-360), UTRs used comprised GalE_BCD12 (“UTR0010_GalE_BCD12”) and GalE_LeuAB (“UTR0014_GalE_LeuAB”) as described by Mutalik et al. (Nat. Methods 2013, No. 10, 354-360), and terminator sequence used was ilvGEDA (“TER0007_ilvGEDA”) as described by Cambray et al. (Nucleic Acids Res. 2013, 41 (9), 5139-5148). All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL minimal medium by diluting 400×. These final 96-well culture plates were then incubated at 37° C. on an orbital shaker at 800 rpm for 72 hours, or shorter, or longer. To measure sugar concentrations at the end of the cultivation experiment whole broth samples were taken from each well by boiling the culture broth for 15 minutes at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 hours at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2SO4 and 20% NH4OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland). The maximum growth speed (mumax) was calculated based on the observed optical densities at 600 nm using the R package grofit.

### Example 3. Production of 6′SL with a Modified E. coli Host

An E. coli K-12 MG1655 strain modified for production of sialic acid as described in Example 2 was transformed with an expression plasmid containing a constitutive transcriptional unit for the N-acylneuraminate cytidylyltransferase (neuA) from P. multocida with UniProt ID A0A849CI62 (SEQ ID NO: 11) and containing a constitutive transcriptional unit for a polypeptide consisting of amino acid residues 108 to 497 of UniProt ID 066375 and having alpha-2,6-sialyltransferase activity with SEQ ID NO: 12 (PdST, reference alpha-2,6-sialyltransferase) or the alpha-2,6-sialyltransferase with SEQ ID NO: 1 (SmuelST). The novel strains were further modified as described in Example 2 to grow on sucrose and use lactose as precursor. The novel strains were evaluated in a 96-well plate for production of 6′SL according to the culture conditions provided in Example 2 in which the strains were cultivated in minimal medium with 30 g/L sucrose and 20 g/L lactose. After 72 hours of incubation, the culture broth was harvested, and the sugars were analyzed on UPLC. For each strain with a particular alpha-2,6-sialyltransferase tested, the measured 6′SL concentration and maximum growth speed was averaged over all biological replicates and then normalized to the averaged 6′SL concentration or maximum growth speed of a reference strain expressing a polypeptide consisting of amino acid residues 108 to 497 of UniProt ID 066375 and having alpha-2,6-sialyltransferase activity (PdST, reference). The experiment showed both alpha-2,6-sialyltransferases were able to produce 6′SL. Surprisingly, and as demonstrated in Table 3, the sialyltransferase with SEQ ID NO: 1 (SmuelST) has a better 6-sialyltransferase binding activity on lactose than the reference sialyltransferase with SEQ ID NO: 12 (PdST).

The experiment also showed that strains expressing the sialyltransferase with SEQ ID NO: 1 (SmuelST) had a higher maximum growth speed compared to strains expressing the reference alpha-2,6-sialyltransferase (SEQ ID NO: 12) (PdST), as is demonstrated in Table 4.

TABLE 3
Relative production of 6′SL (%) in mutant E. coli strains
expressing an alpha-2,6-sialyltransferase and producing sialic
acid, when evaluated in a growth experiment according to the culture
conditions provided in Example 2, in which the culture medium contained
30 g/L sucrose and 20 g/L lactose, and compared to a reference
strain expressing the polypeptide consisting of amino acid residues
108 to 497 of UniProt ID O66375 (SEQ ID NO: 12) (PdST).

| | | 6′SL |
|---|---|---|
| Strain | alpha-2,6-sialyltransferase expressed | (%) |
| A | polypeptide consisting of amino acid residues 108 to 497 | 100 |
| | of UniProt ID O66375 SEQ ID NO: 12 (PdST) | |
| B | SEQ ID NO: 1 (SmuelST) | 170 |

TABLE 4
Relative maximum growth speed (%) in mutant E. coli strains expressing
an alpha-2,6-sialyltransferase and producing sialic acid, when
evaluated in a growth experiment according to the culture conditions
provided in Example 2, in which the culture medium contained 30
g/L sucrose and 20 g/L lactose, and compared to a reference strain
expressing the polypeptide consisting of amino acid residues 108
to 497 of UniProt ID O66375 (SEQ ID NO: 12) (PdST).

| | | mumax |
|---|---|---|
| Strain | alpha-2,6-sialyltransferase expressed | (%) |
| A | polypeptide consisting of amino acid residues 108 to 497 | 100 |
| | of UniProt ID O66375 SEQ ID NO: 12 (PdST) | |
| B | SEQ ID NO: 1 (SmuelST) | 134 |

### Example 4. Production of 6′SL with a Modified E. coli Host

An E. coli K-12 MG1655 strain containing a knock-out of the E. coli lacZ gene is further transformed with an expression plasmid containing a constitutive transcriptional unit for the N-acylneuraminate cytidylyltransferase (neuA) from P. multocida with UniProt ID A0A849CI62 (SEQ ID NO: 11) and containing a constitutive transcriptional unit for a polypeptide consisting of amino acid residues 108 to 497 of UniProt ID 066375 (SEQ ID NO: 12) (PdST, reference alpha-2,6-sialyltransferase) or SEQ ID NO: 1 (SmuelST). The novel strains are evaluated in a 96-well plate for production of 6′SL according to the culture conditions provided in Example 2 in which the strains are cultivated in minimal medium with glycerol as carbon source and sialic acid and lactose as precursors. After 72 hours of incubation, the culture broth is harvested, and the sugars are analyzed on UPLC.

### Example 5. Production of 6′SL with a Modified E. coli Host

An E. coli K-12 MG1655 strain containing a knock-out of the E. coli lacZ gene is further modified for growth on sucrose as described in Example 2. Next, the strain is transformed with an expression plasmid containing a constitutive transcriptional unit for the N-acylneuraminate cytidylyltransferase (neuA) from P. multocida with UniProt ID A0A849CI62 (SEQ ID NO: 11) and containing a constitutive transcriptional unit for a polypeptide consisting of amino acid residues 108 to 497 of UniProt ID 066375 (SEQ ID NO: 12) (PdST, reference alpha-2,6-sialyltransferase) or SEQ ID NO: 1 (SmuelST). The novel strains are evaluated in a 96-well plate for production of 6′SL according to the culture conditions provided in Example 2 in which the strains are cultivated in minimal medium with sucrose as carbon source and sialic acid and lactose as precursors. After 72 hours of incubation, the culture broth is harvested, and the sugars are analyzed on UPLC.

### Example 6. Production of 6′SL with a Modified E. coli Host when Evaluated in a Fed-Batch Fermentation Process with Sucrose and Lactose

The mutant E. coli strain able to produce 6′SL and expressing the alpha-2,6-sialyltransferase with SEQ ID NO: 1 (SmuelST) as described in Example 3 was selected for further evaluation in a fed-batch fermentation process in a 5L bioreactor. Fed-batch fermentations at bioreactor scale were performed as described in Example 2. Sucrose is used as a carbon source and lactose is added in the batch medium as precursor. During fed-batch, sucrose is added via an additional feed. Regular broth samples are taken, and sugars produced are measured as described in Example 2. UPLC analysis of the broth samples taken at different timepoints shows that 6′SL is produced during the fermentation process.

### Example 7. Production of 6′SL with a Modified E. coli Host

An E. coli K-12 MG1655 strain modified for production of sialic acid as described in Example 2 was transformed with an expression plasmid containing a constitutive transcriptional unit for the N-acylneuraminate cytidylyltransferase (neuA) from P. multocida with UniProt ID A0A849CI62 and containing a constitutive transcriptional unit for a P-JT-ISH-224-ST6-like polypeptide consisting of amino acid residues 18 to 514 of UniProt ID A8QYL1 having beta-galactoside alpha-2,6-sialyltransferase activity (P-JT-ISH-224) (SEQ ID NO: 18) or an alpha-2,6-sialyltransferase with SEQ ID NO: 1 (SmuelST). The novel strains were further modified as described in Example 2 to grow on sucrose and use lactose as precursor. The novel strains were evaluated in a 96-well plate for production of 6′SL according to the culture conditions provided in Example 2 in which the strains were cultivated in minimal medium with 30 g/L sucrose and 20 g/L lactose. After 72 hours of incubation, the culture broth was harvested, and the sugars were analyzed on UPLC. For each strain with a particular alpha-2,6-sialyltransferase tested, the measured 6′SL concentration and maximum growth speed was averaged over all biological replicates and then normalized to the averaged 6′SL concentration or maximum growth speed of the strain expressing the P-JT-ISH-224-ST6-like polypeptide consisting of amino acid residues 18 to 514 of UniProt ID A8QYL1 having beta-galactoside alpha-2,6-sialyltransferase activity (P-JT-ISH-224) (SEQ ID NO: 18). The experiment showed both alpha-2,6-sialyltransferases were able to produce 6′SL. Surprisingly, and as demonstrated in Table 5, the sialyltransferase with SEQ ID NO: 1 (SmuelST) has a better 6-sialyltransferase binding activity on lactose than the reference sialyltransferase (P-JT-ISH-224) (SEQ ID NO: 18). The experiment also showed that strains expressing the sialyltransferase with SEQ ID NO: 1 (SmuelST) had a higher maximum growth speed compared to the strain expressing the reference sialyltransferase (P-JT-ISH-224), as is demonstrated in Table 6.

TABLE 5
Relative production of 6′SL (%) in mutant E. coli strains
expressing an alpha-2,6-sialyltransferase and producing sialic
acid, when evaluated in a growth experiment according to the
culture conditions provided in Example 2, in which the culture
medium contained 30 g/L sucrose and 20 g/L lactose, and compared
to a strain expressing the P-JT-ISH-224-ST6-like polypeptide
consisting of amino acid residues 18 to 514 of UniProt ID
A8QYL1 having beta-galactoside alpha-2,6-sialyltransferase
activity (P-JT-ISH-224) (SEQ ID NO: 18).

| | | 6′SL |
|---|---|---|
| Strain | alpha-2,6-sialyltransferase expressed | (%) |
| C | the P-JT-ISH-224-ST6-like polypeptide consisting | 100 |
| | of amino acid residues 18 to 514 of UniProt ID A8QYL1 | |
| | having beta-galactoside alpha-2,6-sialyltransferase | |
| | activity (P-JT-ISH-224) (SEQ ID NO: 18) | |
| B | SEQ ID NO: 1 (SmuelST) | 189 |

TABLE 6
Relative maximum growth speed (%) in mutant E. coli
strains expressing an alpha-2,6-sialyltransferase and
producing sialic acid, when evaluated in a growth experiment
according to the culture conditions provided in Example
2, in which the culture medium contained 30 g/L sucrose
and 20 g/L lactose, and compared to a strain expressing
the P-JT-ISH-224-ST6-like polypeptide consisting of
amino acid residues 18 to 514 of UniProt ID A8QYL1
having beta-galactoside alpha-2,6-sialyltransferase
activity (P-JT-ISH-224) (SEQ ID NO: 18).

| | | mumax |
|---|---|---|
| Strain | alpha-2,6-sialyltransferase expressed | (%) |
| C | the P-JT-ISH-224-ST6-like polypeptide consisting | 100 |
| | of amino acid residues 18 to 514 of UniProt ID A8QYL1 | |
| | having beta-galactoside alpha-2,6-sialyltransferase | |
| | activity (P-JT-ISH-224) (SEQ ID NO: 18) | |
| B | SEQ ID NO: 1 (SmuelST) | 133 |

### Example 8. Materials and Methods Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

### Plasmids

In an example to produce sialic acid and CMP-sialic acid, a yeast expression plasmid was derived from the pRS420-plasmid series (Christianson et al., 1992, Gene 110:119-122) containing the TRP1 selection marker and constitutive transcriptional units for an L-glutamine-D-fructose-6-phosphate aminotransferase like, e.g., the mutant glmS*54 from E. coli (differing from the wild-type E. coli glmS, having UniProt ID P17169 (sequence version 04, 23 Jan. 2007), by an A39T, an R250C and an G472S mutation as described by Deng et al. (Biochimie 88, 419-29 (2006)) (SEQ ID NO: 8), a phosphatase like, e.g., yqaB from E. coli with UniProt ID P77475 (SEQ ID NO: 9), an N-acetylglucosamine 2-epimerase like, e.g., AGE from B. ovatus with UniProt ID A7LVG6 (SEQ ID NO: 6), an N-acetylneuraminate synthase like, e.g., NeuB from N. meningitidis with UniProt ID E0NCD4 (SEQ ID NO: 7) and an N-acylneuraminate cytidylyltransferase like, e.g., NeuA from P. multocida with UniProt ID A0A849CI62 (SEQ ID NO: 11). Optionally, a constitutive transcriptional unit for a glucosamine 6-phosphate N-acetyltransferase like, e.g., GNA1 from S. cerevisiae with UniProt ID P43577 (SEQ ID NO: 5) was added as well. To produce 6′SL, the plasmid further comprised constitutive transcriptional units for a lactose permease like, e.g., LAC12 from K. lactis with UniProt ID P07921 (SEQ ID NO: 13), and one or more sialyltransferase(s) comprising of a polypeptide consisting of amino acid residues 108 to 497 of UniProt ID 066375 and having alpha-2,6-sialyltransferase activity (SEQ ID NO: 12) (PdST, reference alpha-2,6-sialyltransferase) or SEQ ID NO: 1 (SmuelST).

Preferably but not necessarily, any one or more of the glycosyltransferases and/or the proteins involved in nucleotide-activated sugar synthesis were N- and/or C-terminally fused to a SUMOstar tag (e.g., obtained from pYSUMOstar, Life Sensors, Malvern, PA) to enhance their solubility.

Optionally, the mutant yeast strains were modified with a genomic knock-in of a constitutive transcriptional unit encoding a chaperone protein like, e.g., Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5:275). Plasmids were maintained in the host E. coli DH5alpha (F⁻, phi80dlacZdeltaM15, delta(lacZYA-argF) U169, deoR, recA1, endA1, hsdR17 (rk⁻, mk⁺), phoA, supE44, lambda, thi-1, gyrA96, relA1) bought from Invitrogen.

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Gene Expression Promoters

Genes were expressed using synthetic constitutive promoters, as described by Blazeck (Biotechnology and Bioengineering, Vol. 109, No. 11, 2012).

### Example 9. Production of 6′SL with a Modified S. cerevisiae Host

An S. cerevisiae strain is adapted for production of CMP-sialic acid and for expression of a beta-galactoside alpha-2,6-sialyltransferase as described in Example 8 with a yeast expression plasmid comprising constitutive transcriptional units for LAC12 from K. lactis with UniProt ID P07921 (SEQ ID NO: 13), the mutant glmS*54 from E. coli (SEQ ID NO: 8) differing from the wild-type E. coli glmS protein with UniProt ID P17169 (Sequence version 04, 23 Jan. 2007) by an A39T, an R250C and an G472S mutation, the phosphatase yqaB from E. coli with UniProt ID P77475 (SEQ ID NO: 9), AGE from B. ovatus with UniProt ID A7L VG6 (SEQ ID NO: 6), NeuB from N. meningitidis with E0NCD4 (SEQ ID NO: 7) and NeuA from P. multocida with UniProt ID A0A849CI62 (SEQ ID NO: 11), and the polypeptide consisting of amino acid residues 108 to 497 of UniProt ID 066375 and having beta-galactoside alpha-2,6-sialyltransferase activity (SEQ ID NO: 12) (PdST, reference alpha-2,6-sialyltransferase) or the alpha-2,6-sialyltransferase with SEQ ID NO: 1 (SmuelST). When evaluated on SD CSM-Trp drop-out medium comprising lactose as precursor, the mutant yeast strains produce 6′SL. In addition, the beta-galactoside alpha-2,6-sialyltransferase with SEQ ID NO: 1 (SmuelST) has a better 6-sialyltransferase binding activity on lactose than the reference sialyltransferase (SEQ ID NO: 12) PdST.

### Example 10. Evaluation of Alpha-2,6-Sialylltransferase Activity In Vitro

Another example provides the evaluation of alpha-2,6-sialyltransferase activity of the enzyme with SEQ ID NO: 1 of the disclosure in an in vitro enzymatic assay. The enzyme can be produced in a cell-free expression system such as but not limited to the PURExpress system (NEB), or in a host organism such as but not limited to Escherichia coli or Saccharomyces cerevisiae, after which the enzyme can be isolated and optionally further purified. The enzyme extract or purified enzyme is added to a reaction mixture together with CMP-sialic acid and a buffering component such as Tris-HCl or HEPES and a substrate like, e.g., lactose. The reaction mixture is then incubated at a certain temperature (for example, 37° C.) for a certain amount of time (for example, 8 hours, 16 hours, 24 hours), during which the lactose will be converted by the enzyme using CMP-sialic acid to 6′SL. The oligosaccharide is then separated from the reaction mixture by methods known in the art. Further purification of 6′SL can be performed if preferred. At the end of the reaction or after separation and/or purification, the production of 6′SL is measured via analytical methods as described in Example 1 and known by the person skilled in the art.

### Example 11. Materials and Methods Bacillus subtilis

### Media

Two media are used to cultivate B. subtilis: i.e., a rich Luria Broth (LB) and a minimal medium for shake flask cultures. The LB medium consisted of 1% tryptone peptone (Difco), 0.5% yeast extract (Difco) and 0.5% sodium chloride (VWR). Luria Broth agar (LBA) plates consisted of the LB media, with 12 g/L agar (Difco) added. The minimal medium contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose (or another carbon source including but not limited to fructose, maltose, sucrose, glycerol and maltotriose), 10 mL/L trace element mix and 10 mL/L Fe-citrate solution. The medium was set to a pH of 7.0 with 1 M KOH. Depending on the experiment lactose is added as a precursor. The trace element mix consisted of 0.735 g/L CaCl₂·2H₂O, 0.1 g/L MnCl₂·2H₂O, 0.033 g/L CuCl₂·2H₂O, 0.06 g/L CoCl₂·6H₂O, 0.17 g/L ZnCl₂, 0.0311 g/L H₃BO₄, 0.4 g/L Na₂EDTA·2H₂O and 0.06 g/L NazMoO₄. The Fe-citrate solution contained 0.135 g/L FeCl₃·6H₂O, 1 g/L Na-citrate (Hoch 1973 PMC1212887).

Complex medium, e.g., LB, was sterilized by autoclaving (121° C., 21 minutes) and minimal medium by filtration (0.22 μm Sartorius). When necessary, the medium was made selective by adding an antibiotic (e.g., zeocin (20 mg/L)).

### Strains, Plasmids and Mutations

Bacillus subtilis 168 is used as available at the Bacillus Genetic Stock Center (Ohio, USA).

Plasmids for gene deletion via Cre/lox are constructed as described by Yan et al. (Appl. & Environm. Microbial, September 2008, p5556-5562). Gene disruption is done via homologous recombination with linear DNA and transformation via the electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

In an example for sialic acid (Neu5Ac) production, the engineered strain was derived from B. subtilis comprising knockouts of the B. subtilis nagA, nagB and gamA genes and genomic knock-ins of constitutive transcriptional units containing a phosphoglucosamine mutase like, e.g., glmM from E. coli (UniProt ID P31120, Sequence version 03, 23 Jan. 2007), an N-acetylglucosamine-1-phosphate uridyltransferase/glucosamine-1-phosphate acetyltransferase like, e.g., glmU from E. coli (UniProt ID P0ACC7), a UDP-N-acetylglucosamine 2-epimerase like, e.g., neuC from C. jejuni (UniProt ID Q93MP8) and an N-acetylneuraminate synthase like, e.g., neuB from N. meningitidis (UniProt ID E0NCD4). Sialic acid production can also be obtained in modified B. subtilis comprising knockouts of the B. subtilis nagA, nagB and gamA genes and genomic knock-ins of constitutive transcriptional units containing an N-acetylglucosamine 2-epimerase like, e.g., AGE from B. ovatus (UniProt ID A7LVG6) and an N-acetylneuraminate synthase like, e.g., NeuB from N. meningitidis (UniProt ID E0NCD4). To enhance the intracellular glucosamine-6-phosphate pool, the modified strain can further be modified with a genomic knock-in of one or more constitutive transcriptional units containing a glutamine-fructose-6-P-aminotransferase like, e.g., the native glutamine-fructose-6-P-aminotransferase glmS (UniProt ID P0CI73). Optionally, the strains were also modified for expression of a phosphatase like, e.g., SurE from E. coli (UniProt ID P0A840). In an example for sialylated oligosaccharide production, the sialic acid production strains further need to express an N-acylneuraminate cytidylyltransferase like, e.g., neuA from P. multocida with UniProt ID A0A849CI62, and a beta-galactoside alpha-2,6-sialyltransferase like, e.g., a polypeptide consisting of amino acid residues 108 to 497 of UniProt ID 066375 having beta-galactoside alpha-2,6-sialyltransferase activity (PdST) or the polypeptide with SEQ ID NO: 1. Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with a genomic knock-in of a constitutive transcriptional unit for a lactose permease like, e.g., the E. coli LacY (UniProt ID P02920).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72 hours, or shorter, or longer. At the end of the cultivation experiment samples were taken from each well to measure the supernatant concentration (extracellular sugar concentrations, after 5 minutes spinning down the cells), or by boiling the culture broth for 15 minutes at 90° C. or for 60 minutes at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Optical Density, pH and Analytical Analysis

The determination of the optical density and the pH of the bacterial cultures as well as the analytical analysis were performed as described in Example 1.

### Example 12. Production of 6′SL with a Modified B. subtilis Host

A wild-type B. subtilis strain is first modified with genomic knockouts of the B. subtilis genes nagB and gamA together with genomic knock-ins of constitutive transcriptional units for the lactose permease LacY from E. coli (UniProt ID P02920). In a further step, the modified strain is transformed with an expression plasmid comprising constitutive transcriptional units for the N-acylneuraminate cytidylyltransferase neuA from P. multocida (UniProt ID A0A849CI62) and the alpha-2,6-sialyltransferase with SEQ ID NO: 1. The novel strain is evaluated for production of 6′SL when evaluated in a 3-days growth experiment according to the culture conditions provided in Example 11 using appropriate selective medium comprising lactose.

### Example 13. Materials and Methods Corynebacterium glutamicum

### Media

Two different media are used to cultivate C. glutamicum: i.e., a rich tryptone-yeast extract (TY) medium and a minimal medium. The TY medium consisted of 1.6% tryptone (Difco), 1% yeast extract (Difco) and 0.5% sodium chloride (VWR). TY agar (TYA) plates consisted of the TY media, with 12 g/L agar (Difco) added. The minimal medium for the shake flask experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose (or another carbon source including but not limited to fructose, maltose, sucrose, glycerol and maltotriose) and 1 mL/L trace element mix. Depending on the experiment lactose is added as a precursor. The trace element mix consisted of 10 g/L CaCl₂), 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7.0) and 0.03 g/L protocatechuic acid.

Complex medium, e.g., TY, was sterilized by autoclaving (121° C., 21 minutes) and minimal medium by filtration (0.22 μm Sartorius). When necessary, the medium was made selective by adding an antibiotic (e.g., kanamycin, ampicillin).

### Strains and Mutations

Corynebacterium glutamicum ATCC 13032 was used as available at the American Type Culture Collection.

Integrative plasmid vectors based on the Cre/loxP technique as described by Suzuki et al. (Appl. Microbiol. Biotechnol., 2005 April, 67 (2): 225-33) and temperature-sensitive shuttle vectors as described by Okibe et al. (J. Microbiol. Meth. 85, 2011, 155-163) are constructed for gene deletions, mutations and insertions. Suitable promoters for (heterologous) gene expression can be derived from Yim et al. (Biotechnol. Bioeng., 2013 November, 110 (11): 2959-69). Cloning can be performed using Gibson Assembly, Golden Gate assembly, Cliva assembly, LCR or restriction ligation.

In an example for sialic acid production, the engineered strain was derived from C. glutamicum comprising knockouts of the C. glutamicum Idh, cgl2645 and nagB genes and genomic knock-ins of constitutive transcriptional units containing a phosphoglucosamine mutase like, e.g., glmM from E. coli (UniProt ID P31120, Sequence version 03, 23 Jan. 2007), an N-acetylglucosamine-1-phosphate uridyltransferase/glucosamine-1-phosphate acetyltransferase like, e.g., glmU from E. coli (UniProt ID P0ACC7), a UDP-N-acetylglucosamine 2-epimerase like, e.g., neuC from C. jejuni (UniProt ID Q93MP8) and an N-acetylneuraminate synthase like, e.g., neuB from N. meningitidis (UniProt ID E0NCD4). To enhance the intracellular glucosamine-6-phosphate pool, the modified strain can further be modified with a genomic knock-in of one or more constitutive transcriptional units containing a glutamine--fructose-6-P-aminotransferase like, e.g., the native glutamine-fructose-6-P-aminotransferase glmS (UniProt ID Q8NND3, Sequence version 02 (23 Jan. 2007)). In an example for sialylated oligosaccharide production, the sialic acid production strains further need to express an N-acylneuraminate cytidylyltransferase like, e.g., NeuA from P. multocida (UniProt ID A0A849CI62), and a polypeptide consisting of amino acid residues 108 to 497 of UniProt ID 066375 having beta-galactoside alpha-2,6-sialyltransferase activity or the polypeptide with SEQ ID NO: 1. Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with a genomic knock-in of a constitutive transcriptional unit for a lactose permease like, e.g., the E. coli LacY (UniProt ID P02920).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72 hours, or shorter, or longer. At the end of the cultivation experiment samples were taken from each well to measure the supernatant concentration (extracellular sugar concentrations, after 5 minutes spinning down the cells), or by boiling the culture broth for 15 minutes at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g., sialyllactose concentrations, measured in the whole broth by the biomass, in relative percentages compared to the reference strain. The biomass is empirically determined to be approximately ⅓rd of the optical density measured at 600 nm.

### Optical Density, pH and Analytical Analysis

The determination of the optical density and the pH of the bacterial cultures as well as the analytical analysis were performed as described in Example 1.

### Example 14. Production of 6′SL with a Modified C. glutamicum Host

A wild-type C. glutamicum strain is first modified with genomic knockouts of the Idh, cgl2645 and nagB genes together with genomic knock-ins of constitutive transcriptional units for the lactose permease LacY from E. coli (UniProt ID P02920). In a further step, the modified strain is transformed with an expression plasmid comprising constitutive transcriptional units for the N-acylneuraminate cytidylyltransferase neuA from P. multocida (UniProt ID A0A849CI62) and the alpha-2,6-sialyltransferase with SEQ ID NO: 1. The novel strain is evaluated for production of 6′SL when evaluated in a 3-days growth experiment according to the culture conditions provided in Example 13 using appropriate selective medium comprising lactose.

### Example 15. Materials and Methods Chlamydomonas reinhardtii

### Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na₂EDTA·H₂O (Titriplex III), 22 g/L ZnSO₄·7H₂O, 11.4 g/L H₃BO₃, 5 g/L MnCl₂·4H₂O, 5 g/L FeSO₄·7H₂O, 1.6 g/L CoCl₂·6H₂O, 1.6 g/L CuSO₄·5H₂O and 1.1 g/L (NH₄)₆MoO₃.

The TAP medium contained 2.42 g/L Tris (tris(hydroxymethyl)aminomethane), 25 mg/L salt stock solution, 0.108 g/L K₂HPO₄, 0.054 g/L KH₂PO₄ and 1.0 mL/L glacial acetic acid. The salt stock solution consisted of 15 g/L NH₄Cl, 4 g/L MgSO₄·7H₂O and 2 g/L CaCl₂·2H₂O. As precursor for saccharide synthesis, precursors like, e.g., galactose, glucose, fructose, fucose, GlcNAc could be added. Medium was sterilized by autoclaving (121° C., 21 minutes). For stock cultures on agar slants TAP medium was used containing 1% agar (of purified high strength, 1000 g/cm²).

### Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (www.chlamycollection.org), University of Minnesota, U.S.A.

Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from, e.g., Scranton et al. (Algal Res. 2016, 15:135-142). Targeted gene modification (like gene knock-out or gene replacement) can be carried using the Crispr-Cas technology as described, e.g., by Jiang et al. (Eukaryotic Cell 2014, 13 (11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×10⁷ cells/mL. Then, the cells were inoculated into fresh liquid TAP medium in a concentration of 1.0×10⁶ cells/mL and grown under continuous light for 18-20 h until the cell density reached 4.0×10⁶ cells/mL. Next, cells were collected by centrifugation at 1250 g for 5 minutes at room temperature, washed and resuspended with pre-chilled liquid TAP medium containing 60 mM sorbitol (Sigma, U.S.A.), and iced for 10 minutes. Then, 250 μL of cell suspension (corresponding to 5.0×10⁷ cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575 Ω, 50 μFD). After electroporation, the cuvette was immediately placed on ice for 10 minutes. Finally, the cell suspension was transferred into a 50 ml conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23 +−0.5° C. under continuous illumination with a light intensity of 8000 Lx. Cells were analyzed 5 to 7 days later.

In an example for CMP-sialic acid synthesis, C. reinhardtii cells were modified with constitutive transcriptional units for a UDP-N-acetylglucosamine-2-epimerase/N-acetylmannosamine kinase like, e.g., GNE from Homo sapiens (UniProt ID Q9Y223) or a mutant form of the human GNE polypeptide comprising the R263L mutation, an N-acylneuraminate-9-phosphate synthetase like, e.g., NANS from Homo sapiens (UniProt ID Q9NR45) and an N-acylneuraminate cytidylyltransferase like, e.g., CMAS from Homo sapiens (UniProt ID Q8NFW8). In an example for production of sialylated oligosaccharides, C. reinhardtii cells are modified with a CMP-sialic acid transporter like, e.g., CST from Mus musculus (UniProt ID Q61420), and a beta-galactoside alpha-2,6-sialyltransferase like, e.g., from Photobacterium damselae (UniProt ID 066375) or a polypeptide consisting of amino acid residues 108 to 497 of UniProt ID 066375 having beta-galactoside alpha-2,6-sialyltransferase activity (PdST) or the polypeptide with SEQ ID NO: 1.

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

Cells of C. reinhardtii were cultured in selective TAP-agar plates at 23+/−0.5° C. under 14/10 h light/dark cycles with a light intensity of 8000 Lx. Cells were analyzed after 5 to 7 days of cultivation.

For high-density cultures, cells could be cultivated in closed systems like, e.g., vertical or horizontal tube photobioreactors, stirred tank photobioreactors or flat panel photobioreactors as described by Chen et al. (Bioresour. Technol. 2011, 102:71-81) and Johnson et al. (Biotechnol. Prog. 2018, 34:811-827).

### Example 16. Production of 6′SL with Modified C. reinhardtii Cells

C. reinhardtii cells are engineered as described in Example 15 for CMP-sialic acid synthesis with genomic knock-ins of constitutive transcriptional units comprising a mutant form of the UDP-N-acetylglucosamine-2-epimerase/N-acetylmannosamine kinase GNE from Homo sapiens (UniProt ID Q9Y223) differing from the native polypeptide with a R263L mutation, the N-acylneuraminate-9-phosphate synthetase NANS from Homo sapiens (UniProt ID Q9NR45), the N-acylneuraminate cytidylyltransferase CMAS from Homo sapiens (UniProt ID Q8NFW8) and the CMP-sialic acid transporter CST from Mus musculus (UniProt ID Q61420). In a next step, the engineered cells are modified with an expression plasmid comprising a constitutive transcriptional unit comprising the alpha-2,6-sialyltransferase with SEQ ID NO: 1. The novel strain is evaluated for production of 6′SL in a cultivation experiment on TAP-agar plates comprising galactose and glucose as precursors according to the culture conditions provided in Example 15. After 5 days of incubation, the cells are harvested, and the saccharide production is analyzed on UPLC.

### Example 17. Materials and Methods Animal Cells

Isolation of Mesenchymal Stem Cells from Adipose Tissue of Different Animals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% fetal bovine serum), and 1% antibiotics. The culture medium is subsequently replaced with 10% FBS (fetal bovine serum)-supplemented media after the first passage. For example, Ahmad and Shakoori (2013, Stem Cell Regen. Med. 9 (2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 minutes. The cell pellet is washed thrice with phosphate buffer saline and cells are seeded in cell culture flasks in DMEM-F12, RPMI, and Alpha-MEM medium supplemented with 10% fetal bovine serum and 1% antibiotics under standard culture conditions. For example, Hassiotou et al. (2012, Stem Cells. 30 (10): 2164-2174), which is incorporated herein by reference in its entirety for all purposes, describes certain variation(s) of the method(s) described herein in this example.

### Differentiation of Stem Cells Using 2D and 3D Culture Systems

The mesenchymal cells isolated from adipose tissue of different animals or from milk as described above can be differentiated into mammary-like epithelial and luminal cells in 2D and 3D culture systems. See, for example, Huynh et al. 1991. Exp. Cell Res. 197 (2): 191-199; Gibson et al. 1991, In Vitro Cell Dev. Biol. Anim. 27 (7): 585-594; Blatchford et al. 1999; Animal Cell Technology: Basic & Applied Aspects, Springer, Dordrecht. 141-145; Williams et al. 2009, Breast Cancer Res. 11 (3): 26-43; and Arevalo et al. 2015, Am. J. Physiol. Cell Physiol. 310 (5): C348-C356; each of which is incorporated herein by reference in their entireties for all purposes.

For 2D culture, the isolated cells were initially seeded in culture plates in growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48 hours. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24 hours, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48 hours. To induce differentiation, the cells were fed with complete growth medium containing 5 pg/mL insulin, 1 pg/mL hydrocortisone, 0.65 ng/ml triiodothyronine, 100 nM dexamethasone, and 1 pg/mL prolactin. After 24 hours, serum is removed from the complete induction medium.

### Method of Making Mammary-Like Cells

In a next step, the cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodeling is performed using remodeling systems such as CRISPR/Cas9, to activate select genes of interest, such as casein, a-lactalbumin to be constitutively on, to allow for the expression of their respective proteins, and/or to down-regulate and/or knock-out select endogenous genes as described, e.g., in WO21067641, which is incorporated herein by reference in its entirety for all purposes.

### Cultivation

Completed growth media includes high glucose DMEM/F12, 10% FBS, 1% NEAA, 1% pen/strep, 1% ITS-X, 1% F-Glu, 10 ng/mL EGF, and 5 pg/mL hydrocortisone. Completed lactation media includes high glucose DMEM/F12, 1% NEAA, 1% pen/strep, 1% ITS-X, 1% F-Glu, 10 ng/mL EGF, 5 pg/mL hydrocortisone, and 1 pg/mL prolactin (5 ug/mL in Hyunh 1991). Cells are seeded at a density of 20,000 cells/cm2 onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example, the use of nickel resins to remove His-tagged growth factors, to further reduce the levels of contaminants in the lactated product.

### Example 18. Production of 6′SL in a Non-Mammary Adult Stem Cell

Isolated mesenchymal cells and re-programmed into mammary-like cells as described in Example 17 are modified via CRISPR-CAS to express the GlcN6P synthase GFPT1 from Homo sapiens (UniProt ID Q06210), the glucosamine 6-phosphate N-acetyltransferase GNA1 from Homo sapiens (UniProt ID Q96EK6), the phosphoacetylglucosamine mutase PGM3 from Homo sapiens (UniProt ID 095394), the UDP-N-acetylhexosamine pyrophosphorylase UAP1 from Homo sapiens (UniProt ID Q16222), the N-acylneuraminate cytidylyltransferases NeuA from Mus musculus (UniProt ID Q99KK2) and the alpha-2,6-sialyltransferase with SEQ ID NO: 1. Cells are seeded at a density of 20,000 cells/cm2 onto collagen coated flasks in completed growth media and left to adhere and expand for 48 hours in completed growth media, after which the media is switched out for completed lactation media for about 7 days. After cultivation as described in Example 17, cells are subjected to UPLC to analyze for production of 6′SL.

1.-36. (canceled)

37. A method of producing a 6′sialylated disaccharide and/or a 6′sialylated oligosaccharide, the method comprising:
contacting an α-2,6-sialyltransferase comprising a peptide having at least 80.0% sequence identity to SEQ ID NO: 1 over at least 200 amino acids with a mixture comprising a donor comprising a sialic acid residue and an acceptor selected from an oligosaccharide or a disaccharide, under conditions wherein the α-2,6-sialyltransferase catalyzes transfer of a sialic acid residue from the donor to the acceptor, thereby producing the 6′sialylated disaccharide and/or a 6′sialylated oligosaccharide.

38. The method according to claim 37, wherein the peptide has at least 85.0% sequence identity to SEQ ID NO: 1 over a stretch of at least 210 amino acids.

39. The method according to claim 37, having at least 95.0% sequence identity to SEQ ID NO: 1 over 200 amino acids.

40. The method according to claim 37, wherein the α-2,6-sialyltransferase comprises the peptide of SEQ ID NO: 1.

41. The method according to claim 37, wherein the 6′sialylated oligosaccharide is 6′sialyllactose and the acceptor is a disaccharide.

42. The method according to claim 37, the method comprising:
a) providing
i. CMP-sialic acid,
ii. an acceptor, and
iii. the α-2,6-sialyltransferase, wherein the α-2,6-sialyltransferase is an α-2,6-sialyltransferase comprising a peptide having at least 80.0% sequence identity to SEQ ID NO: 1 over at least 200 amino acids,
b) contacting the α-2,6-sialyltransferase and CMP-sialic acid with the acceptor under conditions wherein the α-2,6-sialyltransferase catalyzes the transfer of a sialic acid residue from the CMP-sialic acid to the acceptor resulting in the production of 6′sialylated disaccharide and/or 6′sialylated oligosaccharide,
c) optionally, separating the produced 6′sialylated di- and/or oligosaccharide, and
d) optionally, recovering the 6′sialylated disaccharide and/or 6′sialylated oligosaccharide from cultivation or incubation medium and/or a cell.

43. The method according to claim 37, wherein the method comprises:
contacting a cell extract comprising the α-2,6-sialyltransferase with a mixture comprising a donor comprising a sialic acid residue, and an acceptor comprising an oligosaccharide or disaccharide, under conditions wherein the α-2,6-sialyltransferase catalyzes the transfer of a sialic acid residue from the donor to the acceptor, thereby producing the 6′sialylated disaccharide and/or 6′sialylated oligosaccharide.

44. The method according to claim 37, wherein the 6′sialylated disaccharide and/or 6′sialylated oligosaccharide is produced in a cell-free system.

45. A method for producing a 6′sialylated disaccharide and/or 6′sialylated oligosaccharide, the method comprising:
i. providing a cell expressing an α-2,6-sialyltransferase comprising a peptide having at least 80.0% sequence identity to SEQ ID NO: 1 over at least 200 amino acids, wherein the cell produces CMP-sialic acid, and wherein the cell produces an oligosaccharide or disaccharide,
ii. cultivating and/or incubating the cell under conditions permissive to express the sialyltransferase, the CMP-sialic acid, and the oligosaccharide or disaccharide,
iii. optionally, separating the 6′sialylated disaccharide and/or 6′sialylated oligosaccharide from the cultivation or incubation, and
iv. optionally, recovering the 6′sialylated disaccharide and/or 6′sialylated oligosaccharide from the cell and/or the cultivation or incubation medium.

46. The method according to claim 45, wherein the cell is a metabolically engineered cell.

47. The method according to claim 45, wherein the cultivation medium contains a) at least one carbon source selected from the group consisting of glucose, fructose, sucrose, and glycerol, and/or b) at least one compound selected from the group consisting of lactose, galactose, sialic acid, UDP-galactose (UDP-Gal), and CMP-sialic acid.

48. The method according to claim 37, the method comprising:
i. utilizing a cultivation or incubation medium comprising at least one precursor and/or acceptor for producing the 6′sialylated disaccharide and/or 6′sialylated oligosaccharide or
ii. adding to the cultivation or incubation medium at least one precursor and/or acceptor feed for producing the 6′sialylated disaccharide and/or 6′sialylated oligosaccharide.

49. A cell metabolically engineered to produce a 6′sialylated disaccharide and/or 6′sialylated oligosaccharide, wherein the cell has been metabolically engineered to possess and optionally to express, an α-2,6-sialyltransferase comprising a peptide having at least 80.0% sequence identity to SEQ ID NO: 1 over at least 200 amino acids.

50. The cell of claim 49, wherein the cell is a prokaryotic cell, a eukaryotic cell, a yeast cell, a bacterial cell, an archaebacterial cell, an algae cell, a fungal cell, a bacterium, a fungus, a yeast, a plant cell, an animal cell, or a protozoan cell.

51. The cell of claim 49, wherein the cell comprises a catabolic pathway for selected mono-, di- or oligosaccharides which is at least partially inactivated, the mono-, di-, or oligosaccharides being involved in and/or required for synthesis of a sialylated di- or/and oligosaccharide.

52. The cell of claim 49, wherein the cell produces the 6′sialylated disaccharide and/or 6′sialylated oligosaccharide intracellularly and wherein a fraction or substantially all of the produced 6′sialylated disaccharide and/or 6′sialylated oligosaccharide remains intracellularly and/or is excreted outside the cell.

53. The cell of claim 49, wherein the cell is further genetically modified for:
modified expression of an endogenous membrane protein,
modified activity of an endogenous membrane protein,
expression of a homologous membrane protein, and/or
expression of a heterologous membrane protein,
wherein the endogenous membrane protein is involved in secretion of the 6′sialylated disaccharide and/or 6′sialylated oligosaccharide outside the cell,
optionally, wherein the endogenous membrane protein is selected from the group consisting of porters, P—P-bond-hydrolysis-driven transporters, β-barrel porins, auxiliary transport proteins, putative transport proteins and phosphotransfer-driven group translocators.

54. The cell of claim 49, wherein the cell is further genetically modified for:
modified expression of an endogenous membrane protein,
modified activity of an endogenous membrane protein,
expression of a homologous membrane protein, and/or
expression of a heterologous membrane protein,
wherein the endogenous membrane protein is involved in uptake of a precursor and/or an acceptor for synthesis of the 6′sialylated disaccharide and/or 6′sialylated oligosaccharide,
optionally, wherein the endogenous membrane protein is selected from the group consisting of porters, P—P-bond-hydrolysis-driven transporters, β-barrel porins, auxiliary transport proteins, putative transport proteins and phosphotransfer-driven group translocators.

55. The cell of claim 49, wherein the cell has been modified to reduce production of acetate compared to a non-modified progenitor.

56. The cell of claim 49, wherein the cell has a reduced expression and/or abolished, impaired, reduced or delayed activity of at least one protein comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose: undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIAGlc, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, and pyruvate decarboxylase compared to a non-modified progenitor.

57. The cell of claim 49, wherein the cell is able to produce phosphoenolpyruvate (PEP).

58. The cell of claim 49, wherein the cell is modified for enhanced production and/or supply of phosphoenolpyruvate (PEP) compared to a non-modified progenitor.

59. A vector comprising an isolated nucleic acid molecule encoding an α-2,6-sialyltransferase comprising a peptide having at least 80.0% sequence identity to SEQ ID NO: 1 over at least 200 amino acids.
